SEQUENCE LISTING (61 sequences)

SEQ ID NO: 1 - DNA, 71 bp, Cucumis melo
  POLYMORPHISM (36)..(36): allele G = resistant ; allele A = susceptible
tgatcaccaacaaatttcttggagctgcaagacttragaaagagtgacatttttttttccttcttgagaca

SEQ ID NO: 2 - DNA, 27 bp, Artificial
  Primer
acaaatttcttggagctgcaagactta

SEQ ID NO: 3 - DNA, 26 bp, Artificial
  Primer
caaatttcttggagctgcaagacttg

SEQ ID NO: 4 - DNA, 29 bp, Artificial
  Primer
tatcatcggttcttgtctcaagaaggaaa

SEQ ID NO: 5 - DNA, 71 bp, Cucumis melo
  POLYMORPHISM (36)..(36): allele A = resistant ; allele G = susceptible
ctttggttttcacaccatttgtaagtttgaactttrcaaacactaaagaactcgttatcatacgtgcttgc

SEQ ID NO: 6 - DNA, 28 bp, Artificial
  Primer
ttcacaccatttgtaagtttgaactttg

SEQ ID NO: 7 - DNA, 31 bp, Artificial
  Primer
gttttcacaccatttgtaagtttgaacttta

SEQ ID NO: 8 - DNA, 29 bp, Artificial
  Primer
gcacgtatgataacgagttctttagtgtt

SEQ ID NO: 9 - DNA, 67 bp, Cucumis melo
  POLYMORPHISM (36)..(36): allele G = resistant ; allele A = susceptible
tcgtcttcttcttcttcttctttgttgctatttgargtaaagcggaacaaccctgttttgttattct

SEQ ID NO: 10 - DNA, 25 bp, Artificial
  Primer
gttttgtcccaacaaggcgaaatga

SEQ ID NO: 11 - DNA, 24 bp, Artificial
  Primer
ttttgtcccaacaaggcgaaatgg

SEQ ID NO: 12 - DNA, 29 bp, Artificial
  Primer
gcagaagaagaagaagaagaaacaacgat

SEQ ID NO: 13 - DNA, 69 bp, Cucumis melo
  POLYMORPHISM (34)..(34): allele G = resistant ; allele A = susceptible
atgtaaagaaaagtagatcggcgaaattgtatcrtaatgaactccacaaaactggaaaagatggatgga

SEQ ID NO: 14 - DNA, 20 bp, Artificial
  Primer
ctgaaagcagtttgtgtcga

SEQ ID NO: 15 - DNA, 20 bp, Artificial
  Primer
aaagaaggaagaggctgaga

SEQ ID NO: 16 - DNA, 71 bp, Cucumis melo
  POLYMORPHISM (36)..(36): allele G = resistant ; allele A = susceptible
tctgcaaggcagcctcatcgactgtgaacttcatgrgatagttccagtgaggatcggaaccgccttcttta

SEQ ID NO: 17 - DNA, 23 bp, Artificial
  Primer
aggctaggagtgaccttgataga

SEQ ID NO: 18 - DNA, 22 bp, Artificial
  Primer
ggctaggagtgaccttgatagg

SEQ ID NO: 19 - DNA, 25 bp, Artificial
  Primer
gtcggagtagctgacacttgaagta

SEQ ID NO: 20 - DNA, 71 bp, Cucumis melo
  POLYMORPHISM (36)..(36): allele G = resistant ; allele A = susceptible
agagtgagctgtggaacggagaagctcaaagaaacraagctcgccatgaaagcaacgctacgaagctacga

SEQ ID NO: 21 - DNA, 23 bp, Artificial
  Primer
cgcaacgaaagtaccgctcgaaa

SEQ ID NO: 22 - DNA, 22 bp, Artificial
  Primer
gcaacgaaagtaccgctcgaag

SEQ ID NO: 23 - DNA, 25 bp, Artificial
  Primer
gacaccttgcctcttcgagtttctt

SEQ ID NO: 24 - DNA, 20 bp, Artificial
  Primer
ctggccccctcctaaactaa

SEQ ID NO: 25 - DNA, 21 bp, Artificial
  Primer
caaaaagcatcaaaatggttg

SEQ ID NO: 26 - DNA, 71 bp, Cucumis melo
  POLYMORPHISM (36)..(36): allele A = resistant ; allele G = susceptible
tgggttagaacatacaatcctcatttgggccccggratatcagaacgagtatcagaagccatgagggcaac

SEQ ID NO: 27 - DNA, 18 bp, Artificial
  Primer
cctcatttgggccccggg

SEQ ID NO: 28 - DNA, 21 bp, Artificial
  Primer
aatcctcatttgggccccgga

SEQ ID NO: 29 - DNA, 28 bp, Artificial
  Primer
tcatggcttctgatactcgttctgatat

SEQ ID NO: 30 - DNA, 71 bp, Cucumis melo
  POLYMORPHISM (36)..(36): allele A = resistant ; allele T = susceptible
aagtgacgaagaatattagctgagcctttgatgttwgtatcaatcacattatcaaaatcttgtacatcaat

SEQ ID NO: 31 - DNA, 32 bp, Artificial
  Primer
tacatgttctaaaactattacactaactatgt

SEQ ID NO: 32 - DNA, 32 bp, Artificial
  Primer
tacatgttctaaaactattacactaactatga

SEQ ID NO: 33 - DNA, 29 bp, Artificial
  Primer
gcttcttataatcgactcggaaactacaa

SEQ ID NO: 34 - DNA, 71 bp, Cucumis melo
  POLYMORPHISM (36)..(36): allele A = resistant ; allele G = susceptible
tgtaattaaagtttctgtgtattaaatctgaactcraggcttttcctctatatgtgccttcgaaacgtgct

SEQ ID NO: 35 - DNA, 29 bp, Artificial
  Primer
aaagtttctgtgtattaaatctgaactcg

SEQ ID NO: 36 - DNA, 33 bp, Artificial
  Primer
aattaaagtttctgtgtattaaatctgaactca

SEQ ID NO: 37 - DNA, 25 bp, Artificial
  Primer
cagagcacgtttcgaaggcacatat

SEQ ID NO: 38 - DNA, 26 bp, Artificial
  Primer
cactttctaaatagtttggaaaagag

SEQ ID NO: 39 - DNA, 20 bp, Artificial
  Primer
gagaatgtctctttatctac

SEQ ID NO: 40 - DNA, 20 bp, Artificial
  Primer
cgtagaagacacacataatg

SEQ ID NO: 41 - DNA, 20 bp, Artificial
  Primer
gaactagaaccacaaatcac

SEQ ID NO: 42 - DNA, 47 bp, Cucumis melo
  POLYMORPHISM (36)..(36): allele A = resistant ; allele G = susceptible
atgctagcagtcacagaatttgtagtagacttatarctgaaccattc

SEQ ID NO: 43 - DNA, 29 bp, Artificial
  Primer
cagtcacagaatttgtagtagacttatag

SEQ ID NO: 44 - DNA, 29 bp, Artificial
  Primer
cagtcacagaatttgtagtagacttataa

SEQ ID NO: 45 - DNA, 28 bp, Artificial
  Primer
tcatggcttctgatactcgttctgatat

SEQ ID NO: 46 - DNA, 10921 bp, Cucumis melo
cggagtgtacataataaggagacaccatcacaagtcttgaagctttggcgctcttgaaattcagcttttaaattaattagtcatgtttttcaaatatataacataggagaatatgagttttgcgtcaaatcccatcttggcagagtgtattttgatgaaattccttattggaaggaactgccatatttttagaatttggaatctgtaaagttattctcatgtggttttttgggcagtggagggagttggagaagttgattggtgtgcagatctatgtataggacagatcattgaagaggaaaaaagaattggaatattagggatttggttaattaagcaatatgggaagttggatttcagaattgcaattcatgattgggtttcgatttaggaagaatggacttgcaaattcaatttaacataaaatttcttttttttttcctaaattaatttttatttgatcttcggactcatccaatgcaatttacaggtcactacaaacaatatccatgtcagcacacataacgtgtctagtagacattacgttaacttaatgtaggaaacaaaaagaaaaacgaaaaaggcaagaaatgaaaaaggaaacgttgtgtatgatgattaaaatgtacgttgatagctaaattcaactttgttcttcattagcaacaacaagggacaacaaaatgaaaatttcagcacagttatggaatgttgaagaagaaggtgacgagagagatgccacatcataaaaaattaatattataatcatttttgtcacatcagttttctgattacttatatttaacctacacatcattttgccgttactcaactaacgatcgatatgacctttaaagctcgatgattaaaatgtttattttgaactttcagggactatataaatgtatatatatcaacttcaaatcttagcggtcaaaagtgcattttgttttagtttaaaacaccgagatgtgacgattaataatgcctacacactcacgtactcacacatgcagtgagttttttatgttttttttacacacattagacgcaaattgaattcctaattccattccctaagacaagtttctaatgacttatacaattcacataggcatgcatgcagaacaagcgaaaaacgacgaatttaccttaagaaaatggcccaaccaaccacttacacaatctcaggcataccattccccgggatgaaagaattttgcctgtaattcattcgatctcaggcatacaatgcatcgagaagacgcaaaatgaaagacaaaagtatagaacaacacaatcctgtacttcattttaaacgatagttttcaaatttaacgatcgtgtagatcatgatacacgatctcgagccttagtggcaccgagatggcccgagatgaaagaattatgtctttattttattcgatattaagcatacattacttcgagatgaaagacctctacctttagttattcgatctcgagccttaattgaatcgagatgacccaagattgatagatcgcaaagaagatatatatcgtaagggcatatatgaaatttatgaaaatacgatcatgtgtcataaacttttcttattttgttatatagaccgtaaatattatagattttggttacatttgtgtaaattactctatttttttttagtaaaacaaatgaattaaattttttaaaaaaaaattattgagagttgaagtaattgggtgttttctaaaagattgaaataaatacctttattaactttgcaaatatattttcaaagaggctaacaaatcaaatatattttctaaaaaaatcacattgcgttggggataaggtaagagtaatggaaaaatgaaaggttgtgaacaattcgtactcttacttatgagatttgacagaacaaatatttttcaaatactctttttacgttttcttttttaaaattaaataacaaatacttttccactgcttttcttttcttccattgactcctcaactaataattttggtcttcccattattatcttcttcttcttcatttttctctgcactgtctcttctccatttcaacatcttcttatatcttcattcaggttaaatatttcatcccttaccatttttatttacattttccatctgatcatttctatacttataaactcgataatttgtttagactttctttagatagtttttacttggtgatctgtatgtgttcactcctacttttctgttaggcaaaatcttcaaaattattatggttcattcctcgtggttttttagctcttcaagtatttttgtaggttttgatccttttcaagctcaagaacagaggataataatggacatccttatttcagtcactgcaaaaattgccgaatacactgttgagcctgttggacgccaacttggttatgtatttttcattcgttccaactttcaaaaacttaagactcaagtagaaaagctgaagattacaagagagtctgtgcaacacaagatccatagtgcaagaagaaatgctgaagacataaaacctgccgttgaggaatggttgaaaaaggtcgatgactttgtccgagaatctgacgagatattagccaatgaaggtggacatggtggactctgttccacctatttggtccaacgacacaagttaagtagaaaagcaagcaaaatggtagatgaggttcttgagatgaaaaatgagggggaaagttttgatatggtatcctataaaagtgttatcccatcagttgattgttcacttccaaaagtgcctgactttcttgactttgagtcaagaaagtcgattatggaacaaatcatggatgcactatctgatggtaatgtccataggattggagtatatgggatggggggtgttggcaaaacaatgctagtgaaggatattttaagaaaaattgtggagagtaagaagccttttgatgaggtggtaacatccacgatcagccaaacaccagattttagaagtatccaaggacaactagctgacaagctaggtttgaaattcgaacaagaaacaatagaaggaagggctactattctacgaaagaggttaaagatggagagaagtatcctagttgtgttggatgatgtttgggagtatattgatttggaaactataggaattccaagtgttgaagatcatacggggtgcaagatcttgtttaccactaggattaaacatttgatctcaaatcaaatgtgcgccaataaaatttttgagataaaagttttaggaaaagatgagtcatggaatttatttaaggcaatggcaggtgacattgttgatgcaagtgatttgaagcctatagccattcgaattgtgagagaatgtgcaggtttgcctattgctattactactgttgctaaggcattacgaaataaaccttccgacatttggaatgatgccttagatcagcttaaaactgttgatgtgggtatggcaaacattggagaaatggaaaagaaagtgtatttgtcactaaaactgagttacgattgcttgggatatgaagaggtgaagttattattcttgttatgcagcatgtttccagaagactttagcattgacgtggaagggttgcatgtatatgccatgggcatgggattcttacatggtgttgatactgtggtaaaaggacgacgtaggataaaaaaattggttgatgatcttatatcttcttctttgcttcaacaatattctgagtatgggtgcaattatgtgaaaatgcatgatatggttcgtgatgtagccctattaattgcatctaagaatgaacacgtacgtacattgagctatgtgaaaagatcgaatgaagaatgggaagaagagaaactattgggtaatcataccgcagtgttcattgatggtttacattatcctctcccgaagttaacgttacccaaagttcaattattaaggttagttgcaaaatattgttgggaacataataagcgtgtgtcggtggtagaaactttttttgaagaaatgaaagagctcaaaggtttagtagtagaaaacgtaaatatatcattgatgcaacgaccatctgatgtttactccttagcaaacatcagagtattacgtttggaaagatgtcaattattagggagcatagattggattggtgaattaaaaaagcttgaaattcttgattttagtgaatctaacatcacacaaattcctacaaccatgagccaattgacacagctaaaagtgttgaatttatcttcttgtgaacaacttgaggtaattccaccaaatattctttcaaagttgacaaaattggaagaattagatctggaaacttttgatggatgggaaggagaagaatggtatgaaggaaggaaaaatgctagcctttctgaactcaagtgcttgcgacacctttatgctttaaacttaaccattcaagatgaagaaattatgccagaaaacttgttcttagttgggaagttgaagcttcaaaaattcaacatttgtattggttgcgaaagcaaattaaagtatacttttgcatacaagaacagaatcaaaaacttcattggaatcaagatggaatcaggaaggtgcttggatgattggataaaaaatttgttaaagaggtcggacaatgtgcttttggaaggatcagtttgttcaaaggttctccactcagaattggtaggtgccaataacttcgtaagtttgcagtatctctacctttatgataattcaaaatttcaacattttatcaacgttagcaataccatcaacattgaagaatcattttttagtgaaatggtaaatttcatcaacacatttttctatgtttctatatacacatcaaaatttataaatcacctgtttgttcatttttattgttccttccacaagttccttctaacatttgtggttagtaatgatattagggataagattctattcttctttgttttagaaggctcttctcttggtgtagagagactctttcttgtttcatacattaatgataatatataattttagacttctaaaatatataaatgcatgtcaggtgatattccaagaaaatttaaagaagtttattagatatttcctatctaaggaagcaaatttgaagtaatcattttacacaatttgttatggtaattttaaaacactatataaattatatgttgttagttagcatcagttgtgatgatatggttaattacgttgtcacaaactgaacttttaaggacgtaaatttgtaggtatcacttcctaatttggagaagttggaaattgtgaatgcaaagagtttgaagatgatatggagcaataacgtgccaattcttaattccttttccaaactcgaggaaataaaaatttattcatgcaacaatcttcaaaaagtattatttcctccaaatatgatggacattctcacatgccttaaagtcttagagatcaaaaattgtgatttgttggaagggatatttgaagcgcaagagccaattagtgttgttgagagcaataatttacccattcttaattccttttccaaactcgaggaaataagaatttggtcatgcaacaatcttcaaaaagtattatttccttcaaatatgatgggcattcttccatgccttaaagtcttagatattagaggttgtgaattgttggaagggatatttgaagtgcaagagccaattagtgttgttgagagcaatagtgtacccattcttaattccttttccaaactcgagaaaataagaatttggtcatgcaacaatcttcaaaaaatattatttccttcaaatatgatgggcattcttacatgccttaaagtcttagagatcagagattgtgaattgttggaagggatatttgaagtgcaagagccaattagtgttgttgagagcaataatttacccattcttaattccttttccaaactcgaggaaataagaattgggtcatgcaacaatcttcaaaaagtattatttcctccaaatatgatgggcattcttacatgccttaaagtcttagagattagacattgtaatttgttggaagggatatttgaagtgcaagagccaattagtattgttgaagcgagtcctatcttgctccaaaatttatcttcgttgatgttatgtaatcttccaaaccttgagtacgtgtggagcaaaaatccttatgaacttctgagtttggaaaatataaaaagtttgaccattgataaatgtccaagacttagaagagaatactcagtcaaaattctaaagcaacttgaagatgtaagcatagatatcaaacaattgatgaaggttattgagaaggaaaagtcagcacatcataatatgttggaatcaaagcaatgggagacttcatcttcttctaaggtacgtatatattctacaagaaaacatgtttgttcaatttaattttcagaaaataaattgttttcaagaattagttgaacagaattggttctgttagttgaacagagttttaaagaaaacaaattattatgtttgttctagaaacctatggaaattagtgttattaggggttaattatttacctgaattaaaaaagaataagcaagtaaaccgcttaattttaatatgtttatcaatagaaattaggatccgtttggattgacttgaatgatatgttttcctggaaagaaactcatttttgtttgaactcatttttatgaaaattggctaaaatatatttaaaaaattaggtggctttcaaatattcaattttttttttaaaataacttattttttgaattaaacactccaaaatgtaattcaaaaacacccctaagttaattagatatataattaatacataaattaattaattttatttagttgagttaaattagttttaatgcaaacaataatatatttttatagccaatatacaagaatgaaagtaagaaaagaagacaaaaaaaaatgatatgttaattagaattttacataaagaatgatctgattaaaagctaatctctatcgatatagttttttaattatattaaatatagtaagtagtccattataataattgaatttttttgtaccaaattaattaaactaattctttttactaattattgaatatttaagatatttttggctaattaattaataatttgtgtgcaaatataagttattattgcatttatttaatagattttgatcatcaggatggggttctacggctgggagatggttctaagttgtttccaaatcttaaaagtttgaagctatatggttttgttgattataactcaacccatttaccaatggaaatgttgcaaatcttattccaacttgtagtctttgaattggaaggagcatttcttgaagaaattttccccagcaacatactgattccaagctatatggttttaagaagattagctctatctaaactacccaagcttaagcatttgtggagtgaagaatgctcacaaaacaatatcacctcagttcttcaacatttgatttctctaagaatttcagaatgtggaagattgagtagtttactgtcgtcaatagtgtgttttacaaacttgaaacatcttcgggtttataaatgtgatggactaacccatttgctgaatccttcggtggctacaacgcttgtgcaacttgagtctttgacaatagaagaatgcaaaaggatgagtagtgtaattgagggaggatcaaccgaagaagatggaaatgatgaaatggttgtattcaacaacctacaacatttatacatttttaattgttccaacctaacaagcttttattgtgggagatgcattattaaatttccatgtttgaggcaagtagacatttggaactgttctgaaatgaaggtcttttcgcttggaattgtaagcacacctcgattgaaatatgaaaatttttctttaaagaatgattacgatgatgaacggtgtcatccaaaatatcccaaagatatgttggtggaagatatgaatgtcatcaccagagaatattgggaggataatgttgataccggaattccaaatttatttgccgaacaggtttgtatatttaattaccttttcatatttggtaataattaatttttattatttgtgtgttagagtatgaactttaatgaatttatttaattaatgcagagtttggaggaaaaccgatctgaaaattcttcttcttcgaagaataatgttgagaaagaataaggaattatatggatattgttgtacactacttaatatatcatttcatccacaaggaaaaggtcagaatcttgaaatcctccattcttttttatgagagaatatcatccaatgtcaaattgaaaagtctcgatagatttgttaaattaacttttgatacaagtcataaaatgttaattagtataataataatatatctgatcccatcaaattaattagaagtaacgacaaatttaacttctgtaatatcaattcaatttgatgtctcaatcaactgcataaaatttgatgtcacaaatttaacttctgtaatgtgaatcttttttttttttccctttgcacataaaaccaacaagttaaaatagatataacaaagaatttaattcacatataataaattcatccaatataatgttctttcacctttttctctctttcacaaaactgtaataataatatctaccacaaaaggtaaaccattaatatgattcttcaagaaggttgtttatttggtcaaattttcatgaaagtattaatacagtgtatgttttgcaaaggaagctgccaaatacctacctcaatcctagcagatgcgttctttgcaagttggctgtcaaagacttgaaccatattttcacaacctgtgccaattcgcaaatagcctctgggtcaaactgcacgacaaaattggtggaaactttgatacaaacagtatcaaagccctctgttggtctcttggctcgctgaaacaatcaaacagaaagaacatcattctctcgatgtaggtgtggatcttctttagtccatttgggtggaaagaaacaataggattttcatagacacagaaagaagcttagtcacatttgggaagatatcgaaactttgattggaccatggtcgagtagaaacaaaatgttcaaagactacaatccaacatcaatctttaaactttagagctttgttagattaatgtttgttgtatatggcttccctgtagccaaagaaatatacattgtaacaatggtttgatgaaatgataatgaagtggtatggtgtgtttcaattgcaaaaaatatgattcttcaagaaaggccgagaggataagattgtgatcgtgccatgcgcttgggttgtgatttaattacaaatatattcatatagtatttggagcaaaacagtcctaattaattaaatataatgcgttattttatttttctgaataagttaaatttaaccactagaaatttttttactaagtgaacattttcatagcactttcataatccccacttcattaaaatgaatcaataaagttcaaggaaagtgtagaatgatgaatctaaaagaaacaaaaaacaaggtatataagtttaaagcaacggtggtagcactgtaatgatccgagggagtacacattttttcataaattattttattatcatcaatagaagaattcgaatttcttttatcttttaattgataatataatttcataattagctataagtggatttttcaaatgattggatccacatacatatcaacttcgatcaaattattaaaggtataattttaaatcactaagaagaaaaggattgatggtgggccttatgctattatacactttgagtaccctctcggcctcaacaagattcccaagttcaataattgaaactttgaaaaatgtgtgcaatcacatgtattcaatttctatgtcggtatcacatattttcatgtttccatacgtttaatttccatataaaagtgaatccataattttattatattgcgaaaaaatctaccaaacatggaaattagctatgaaaatattagtaatataaataatggatatgttgacttatttgaaaaaaattaaaaaaatattcatttatgggtttaaatttatttttaaattttttgtttttatatattattttaaaaatatatattgaaattaatattttattgatattatatcatcaaaatttttgtaaaattaagatcattaatataacgccccagacccaagatttggaattcggatccctgacattcttttgcatccactgtgatctgataacatcatctttacttgtcttaaattattagactgaaagttctctccacaaaacaataggagtcatttcaacatactttgtcctcactcacagcatccctatcactcataattaatgtaaaattttattatatttgtaaatatgttttggtgtactttgccatatattaaaacatactactaaacaaaacgaaatgattaaaaaggaagggaaggtattaaaaattataaattttaagaaaggaaaagaagaagaaggaaaaaaaaaaaaaaaagagaatgatgagtgagaggcaccaagtgaggacatatactactctttgagtacataacctaatggttaagaaaaaaaaaaatctcatatcaaattcaaagtgccatgctattattacttaatattttatatggaagttaaataaattgttagagagaagtcttgttttctgtctgtttgttaactcatttttgtaattaatgtttaatttgatcattgtcattccaattaattgtaacataattttctgcccaatttatctcttttgctttcgtttttgtttagataccctactcggctactcctcaacttttcctcatttcattttcagttgg

SEQ ID NO: 47 - protein, 1467 aa, Cucumis melo
MDILISVTAKIAEYTVEPVGRQLGYVFFIRSNFQKLKTQVEKLKITRESVQHKIHSARRNAEDIKPAVEEWLKKVDDFVRESDEILANEGGHGGLCSTYLVQRHKLSRKASKMVDEVLEMKNEGESFDMVSYKSVIPSVDCSLPKVPDFLDFESRKSIMEQIMDALSDGNVHRIGVYGMGGVGKTMLVKDILRKIVESKKPFDEVVTSTISQTPDFRSIQGQLADKLGLKFEQETIEGRATILRKRLKMERSILVVLDDVWEYIDLETIGIPSVEDHTGCKILFTTRIKHLISNQMCANKIFEIKVLGKDESWNLFKAMAGDIVDASDLKPIAIRIVRECAGLPIAITTVAKALRNKPSDIWNDALDQLKTVDVGMANIGEMEKKVYLSLKLSYDCLGYEEVKLLFLLCSMFPEDFSIDVEGLHVYAMGMGFLHGVDTVVKGRRRIKKLVDDLISSSLLQQYSEYGCNYVKMHDMVRDVALLIASKNEHVRTLSYVKRSNEEWEEEKLLGNHTAVFIDGLHYPLPKLTLPKVQLLRLVAKYCWEHNKRVSVVETFFEEMKELKGLVVENVNISLMQRPSDVYSLANIRVLRLERCQLLGSIDWIGELKKLEILDFSESNITQIPTTMSQLTQLKVLNLSSCEQLEVIPPNILSKLTKLEELDLETFDGWEGEEWYEGRKNASLSELKCLRHLYALNLTIQDEEIMPENLFLVGKLKLQKFNICIGCESKLKYTFAYKNRIKNFIGIKMESGRCLDDWIKNLLKRSDNVLLEGSVCSKVLHSELVSLPNLEKLEIVNAKSLKMIWSNNVPILNSFSKLEEIKIYSCNNLQKVLFPPNMMDILTCLKVLEIKNCDLLEGIFEAQEPISVVESNNLPILNSFSKLEEIRIWSCNNLQKVLFPSNMMGILPCLKVLDIRGCELLEGIFEVQEPISVVESNSVPILNSFSKLEKIRIWSCNNLQKILFPSNMMGILTCLKVLEIRDCELLEGIFEVQEPISVVESNNLPILNSFSKLEEIRIGSCNNLQKVLFPPNMMGILTCLKVLEIRHCNLLEGIFEVQEPISIVEASPILLQNLSSLMLCNLPNLEYVWSKNPYELLSLENIKSLTIDKCPRLRREYSVKILKQLEDVSIDIKQLMKVIEKEKSAHHNMLESKQWETSSSSKDGVLRLGDGSKLFPNLKSLKLYGFVDYNSTHLPMEMLQILFQLVVFELEGAFLEEIFPSNILIPSYMVLRRLALSKLPKLKHLWSEECSQNNITSVLQHLISLRISECGRLSSLLSSIVCFTNLKHLRVYKCDGLTHLLNPSVATTLVQLESLTIEECKRMSSVIEGGSTEEDGNDEMVVFNNLQHLYIFNCSNLTSFYCGRCIIKFPCLRQVDIWNCSEMKVFSLGIVSTPRLKYENFSLKNDYDDERCHPKYPKDMLVEDMNVITREYWEDNVDTGIPNLFAEQSLEENRSENSSSSKNNVEKE

SEQ ID NO: 48 - DNA, 24 bp, Artificial
  Primer
ctccactcagaattggtaggtgcc

SEQ ID NO: 49 - DNA, 24 bp, Artificial
  Primer
ccttagaagaagatgaagtctccc

SEQ ID NO: 50 - DNA, 71 bp, Cucumis melo
  POLYMORPHISM (36)..(36): allele A = resistant ; allele C = susceptible
tgagctcatgagacgaagcgtttgattcatgtgacmccgggtttccaagagtaacaatttctcgagcttta

SEQ ID NO: 51 - DNA, 46 bp, Artificial
  Primer
gaaggtgaccaagttcatgctgacgaagcgtttgattcatgtgaca

SEQ ID NO: 52 - DNA, 45 bp, Artificial
  Primer
gaaggtcggagtcaacggattacgaagcgtttgattcatgtgacc

SEQ ID NO: 53 - DNA, 30 bp, Artificial
  Primer
ctaaagctcgagaaattgttactcttggaa

SEQ ID NO: 54 - DNA, 71 bp, Cucumis melo
  POLYMORPHISM (36)..(36): allele A = resistant ; allele G = susceptible
aatgaattagtcgatcgagtgagcatggatgggaartttctacaggaatcactatccagaactaaaaaagg

SEQ ID NO: 55 - DNA, 45 bp, Artificial
  Primer
gaaggtcggagtcaacggattctggatagtgattcctgtagaaac

SEQ ID NO: 56 - DNA, 48 bp, Artificial
  Primer
gaaggtgaccaagttcatgctgttctggatagtgattcctgtagaaat

SEQ ID NO: 57 - DNA, 27 bp, Artificial
  Primer
gaattagtcgatcgagtgagcatggat

SEQ ID NO: 58 - DNA, 71 bp, Cucumis melo
  POLYMORPHISM (36)..(36): allele G = resistant ; allele A = susceptible
tctctacattagatttcccttcatgatgttgtagtracatggagggttgtttcctttaaaaatgtacaggt

SEQ ID NO: 59 - DNA, 50 bp, Artificial
  Primer
gaaggtgaccaagttcatgctattagatttcccttcatgatgttgtagta

SEQ ID NO: 60 - DNA, 47 bp, Artificial
  Primer
gaaggtcggagtcaacggattagatttcccttcatgatgttgtagtg

SEQ ID NO: 61 - DNA, 30 bp, Artificial
  Primer
tgtacatttttaaaggaaacaaccctccat

(19) United States
(12) Patent Application Publication
(10) Pub. No.: US 2023/0276759 A1
(43) Pub. Date: Sep. 7, 2023

(54) MELON PLANTS RESISTANT TO SCAB DISEASE, APHIDS AND POWDERY MILDEW

(71) Applicant: VILMORIN & CIE, Paris (FR)

(72) Inventors: Michael MOREAU, Saint Remy de Provence (FR); Frank DE LANGEN, Saint Remy de Provence (FR); Jamila CHAIB, La Mojonera (ES)

(73) Assignee: VILMORIN & CIE, Paris (FR)

(21) Appl. No.: 18/008,392

(22) PCT Filed: Jun. 2, 2021
(86) PCT No.: PCT/EP2021/064790

§ 371 Date: Dec. 5, 2022

(30) Foreign Application Priority Data
Jun. 3, 2020 (WO) PCT/IB2020/000519

(51) Int. Cl. A01H 1/04; A01H 5/08; A01H 6/34; C12Q 1/6895

(52) U.S. Cl. CPC A01H 1/04; A01H 5/08; A01H 6/346; C12Q 1/6895; C12Q 2600/13; C12Q 2600/158

(57) ABSTRACT

The present invention relates to Cucumis melo (C. melo) plants with resistance to Scab disease, aphids and Powdery Mildew (PM) combined with desirable agronomic traits. The present invention also provides methods of making such plants, and to methods of detecting and/or selecting such plants.

The present invention relates to Cucumis melo (C. melo) plants with resistance to Scab disease, aphids and Powdery Mildew (PM) combined with desirable agronomic traits. The present invention also provides methods of making such plants, and methods of detecting and/or selecting such plants.

Many pathogens, such as Cladosporium, Podosphaera xanthii or aphids, can colonize cultures of melon (Cucumis melo).

Cladosporium cucumerinum Ellis and Arthur is a fungus that causes the Scab disease attacking the foliage and fruits of several cucurbits, e.g. cucumber and melon, in many parts of the world. Monogenic resistance at very high level has been introduced systematically in cucumber last fifty years but no resistance has been described in melon. The disease occurs mainly in cool, wet conditions, and it is widespread for example in the south-west of France where producers regularly use fungicides.

Powdery mildew (PM) is a foliar disease that is caused by two main agents: Golovinomyces cichoracearum and Podosphaera xanthii. Podosphaera xanthii is predominant in most countries, whereas Golovinomyces cichoracearum can cause disease in temperate zones. In France, despite the presence of the two agents, Podosphaera xanthii is the species most commonly found. On melon, five races of Podosphaera xanthii (i.e. races Px-1, Px-2, Px-3, Px-5, Px-3-5) and two races of Golovinomyces cichoracearum have been described.

Melon aphid, i.e. Aphis gossypii Glover, is an insect pest that colonizes a wide range of economically important host plants, such as cucurbits, for which it is the major pest. Colonization of cucurbits by A. gossypii causes stunting and severe leaf curling that can result in plant death. A. gossypii is also an efficient virus vector and thus contributes to the spreading of viral diseases. Resistant melon accessions have been described since 1970s, and a major gene responsible for the resistance has been identified, i.e. the Vat gene (Dogimont et al., Cucurbitaceae 2008, Proceedings of the IXth EUCARPIA meeting on genetics and breeding of Cucurbitaceae; Dogimont et al., 2014, The Plant Journal, 80, 993-1004), and introgressed into commercial melon lines. However, it has been demonstrated that the Vat gene is linked with a necrotic reaction (Flaccida necrosis) (Pitrat and Lecoq, 1982, Agronomie, 2:503-508).

To date, resistance to several races of Podosphaera xanthii and the aphid Aphis gossypii are currently present in Charentais varieties grown in France. Two independent loci, PmV.1 and PmXII.1, conferring resistance to races 1, 2, 3 and 5 of Podosphaera xanthii have been identified using a RIL population derived from a cross between the resistant melon accession PI 124112 and the susceptible melon “Vedrantais” by Perchepied et al., 2005, The American Phytopathological Society, Vol. 95(5):556-565. Moreover, in Fukino et al., 2008, Theor. Appl. Genet., 118(1):165-75, two QTLs conferring PM resistance to Podosphaera xanthii races A and B were detected on linkage group (LG) II (or LG2) and LGXII (LG12) by using RILs derived from the crosses between the resistant AR5 cantaloupe breeding line and the susceptible Japanese cultivar “Earl's Favourite (Harukei 3)”. The QTL on LGII in this study has been found to be closely linked to the CMBR8 and CMBR120 markers.

Fazza et al., 2013, Crop Breeding and Applied Biotechnology, 13:349-355, also disclose the mapping of QTLs conferring resistance to races 1, 3 and 5 of Podosphaera xanthii in the melon accession PI 414723 on two linked loci in LGII (LG2). However, the PI 414723 accession is an Indian accession with undesirable agronomic traits, such as having pale, mealy and soft flesh at maturity, a big cavity, high yellowing/orange rind at maturity and low level of sugar (6° Brix) (Burger et al., 2010, Horticultural Reviews, 36, 165-198). Fazza et al. also disclose that the LGII contains other disease resistance gene, such as the Zym gene that confers resistance to the Zucchini Yellow Mosaic Virus (ZYMV). However, a leaf/stem/fruit necrotic streak phenotype has been observed on Zym/Zym homozygous plants (Pitrat and Lecoq, 1984, Euphytica, 33(1):57-61, US20140059712). There is thus a need to obtain plants having the resistance to PM without the deleterious necrotic effect associated to the presence of the Zym gene.

Moreover, the CMBR8 marker identified in Fukino et al. is not mapped in Fazza et al. and the CMBR120 marker is quite distant from their QTL identified on LGII, suggesting that the QTL of Fukino et al. and the QTL of Fazza et al. are located in different portion of the LGII. Anyway today due to the low density of genetically mapped marker in the melon genome, it is difficult to know the precise location of such QTL of resistance. There is thus a diversity of QTLs of resistance to different races of Podosphaera xanthii, for which a precise location is needed in order to define whether they are potentially combinable, and then be able to combine them in one melon plant.

With the reduction of plant protection products used for the disinfection of floors, one can expect an increase in the incidence of all diseases related to the ground. The development of multi-resistant cultivar, such as cultivar resistant to Cladosporium, Podosphaera xanthii and aphids, is also thus needed to have more acceptable and economic means for controlling the impact of pathogens.

### SUMMARY OF THE INVENTION

The present inventors have been able to introgress in Cucumis melo (C. melo) plants quantitative trait loci conferring resistance to Scab disease, aphids and Powdery Mildew (PM), combined with desirable agronomic traits and preferably without any necrotic phenotype linked to the Zym gene. Thus, in a first embodiment, the present invention provides a C. melo plant that is resistant to Scab disease, aphids and Powdery Mildew (PM), wherein said plant:
comprises:
   (i) at least one QTL conferring resistance to Scab disease, wherein said at least one QTL is present on linkage group (LG) 2 (LGII) and/or linkage group 5 (LG5 or LGV),
   (ii) at least one QTL conferring resistance to PM, wherein said at least one QTL is present on LG2 (LGII) and/or LG5 (LGV), and is different from said at least one QTL in (i), and
   (iii) the Vat gene analog conferring resistance to aphids on LG5 (LGV), and
has a commercially acceptable fruit quality.
   Preferably, such a plant does not have any necrotic phenotype linked to the Zym gene.

In some embodiments, said QTL conferring resistance to Scab disease that is present on LG2 is located within a chromosomal region that is delimited by marker Cm_MU45136_209 (also named MU45136_209, SEQ ID NO: 1) and marker Cm_MU45398_32 (also named MU45398_32, SEQ ID NO: 9).

In some embodiments, said QTL conferring resistance to Scab disease that is present on LG5 is located within a chromosomal region that is delimited by marker LG5-M1 (SEQ ID NO: 13) and marker Cm_MU44050_58 (also named MU44050_58, SEQ ID NO: 20).

In some embodiments, said QTL conferring resistance to PM that is present on LG2 is located within a chromosomal region that is delimited by marker CMBR120 (which can be identified by using the primers having SEQ ID NO: 24 and 25) and marker Cm_MU47536_461 (also named MU47536_461, SEQ ID NO: 30).

In some embodiments, said QTL conferring resistance to PM that is present on LG5 is located within a chromosomal region that is delimited by marker Cm_MU45437_855 (also named MU45437_855, SEQ ID NO: 34) and marker LG5-M3 (SEQ ID NO: 42).

In some embodiments, the C. melo plant that is resistant to Scab disease, aphids and PM is the line MTYVVC721, for which a representative sample of seeds have been deposited at the NCIMB under the accession number NCIMB 43317.

Also provided are cells of a Cucumis melo (C. melo) plant according to the invention.

Further provided is a plant part obtained from a C. melo plant according to the invention. In some embodiments, said plant part is a seed, a fruit, a reproductive material, roots, flowers, a rootstock or a scion.

The present invention also provides seeds of a Cucumis melo (C. melo) plant, giving rise when grown up to a plant according to the invention.

Also provided are hybrid plants of Cucumis melo (C. melo) resistant to Scab disease, aphids and Powdery Mildew (PM), obtainable by crossing a C. melo plant with a resistant plant according to the invention.

The present invention also provides methods for detecting and/or selecting a Cucumis melo (C. melo) plant that is resistant to Scab disease, aphids and powdery mildew (PM), wherein said method comprises the steps of:
a) detecting the presence or absence of:
   (i) at least one QTL conferring resistance to Scab disease, said at least one QTL being present on linkage group (LG) 2 and/or linkage group 5 (LG5),
   (ii) at least one QTL conferring resistance to PM, said at least one QTL being present on LG2 and/or LG5, and being different from said at least one QTL in (i), and
   (iii) the Vat gene analog conferring resistance to aphids on LG5,
b) selecting as a plant resistant to Scab disease, aphids and powdery mildew (PM), the C. melo plant in which said at least one QTL conferring resistance to Scab disease, said at least one QTL conferring resistance to PM, and said Vat gene analog conferring resistance to aphids have been detected as present.

Further provided is the use of one or more markers for detecting Cucumis melo (C. melo) plant that is resistant to Scab disease, aphids and powdery mildew (PM), wherein said one or more markers is/are located in at least one of the following chromosomal regions:
in the chromosomal region delimited on LG2 by marker Cm_MU45136_209 and marker Cm_MU45398_32,
in the chromosomal region delimited on LG5 by marker LG5-M1 and marker Cm_MU44050_58,
in the chromosomal region delimited on LG2 by marker CMBR120 and marker Cm_MU47536_461,
in the chromosomal region delimited on LG5 by marker Cm_MU45437_855 and marker LG5-M3, or
in the Vat gene analog.

The present invention also provides the use of the Cucumis melo (C. melo) resistant plant according to the invention as a breeding partner in a breeding program for obtaining C. melo plants resistant to Scab disease, aphids and Powdery Mildew (PM) and that preferably does not have any necrotic phenotype linked to the Zym gene.

Also provided are methods for producing Cucumis melo (C. melo) seeds. In some embodiments, the methods comprise crossing the Cucumis melo (C. melo) plant according to the invention with itself or with another C. melo plant, and harvesting the resultant seeds.

Further provided is a method for increasing the number of harvestable Cucumis melo (C. melo) plants in an environment infested by Scab disease, aphids and powdery mildew comprising growing in said environment C. melo plants resistant to Scab disease, aphids and Powdery Mildew (PM) which:
comprises:
(i) at least one QTL conferring resistance to Scab disease, wherein said at least one QTL is present on linkage group (LG) 2 and/or linkage group 5 (LG5),
(ii) at least one QTL conferring resistance to PM, wherein said at least one QTL is present on LG2 and/or LG5, and is different from said at least one QTL in (i), and
(iii) the Vat gene analog conferring resistance to aphids on LG5, and
   has a commercially acceptable fruit quality.
      Preferably, such a plant does not have any necrotic phenotype linked to the Zym gene.

Also provided is a method for protecting a field from infestation and/or spread of Scab disease, aphids and Powdery Mildew (PM), comprising growing C. melo plants resistant to Scab disease, aphids and PM which:
comprises:
(i) at least one QTL conferring resistance to Scab disease, wherein said at least one QTL is present on linkage group (LG) 2 and/or linkage group 5 (LG5),
(ii) at least one QTL conferring resistance to PM, wherein said at least one QTL is present on LG2 and/or LG5, and is different from said at least one QTL in (i), and
(iii) the Vat gene analog conferring resistance to aphids on LG5, and
   has a commercially acceptable fruit quality, and
   preferably does not have any necrotic phenotype linked to the Zym gene.

The use of a C. melo plants resistant to Scab disease, aphids and Powdery Mildew (PM) which:
comprises:
(i) at least one QTL conferring resistance to Scab disease, wherein said at least one QTL is present on linkage group (LG) 2 and/or linkage group 5 (LG5),
(ii) at least one QTL conferring resistance to PM, wherein said at least one QTL is present on LG2 and/or LG5, and is different from said at least one QTL in (i), and
(iii) the Vat gene analog conferring resistance to aphids on LG5, and
   has a commercially acceptable fruit quality, and
   preferably does not have any necrotic phenotype linked to the Zym gene, for controlling infestation in a field by Scab disease, aphids and PM is also provided.

Further provided is a method for the production of C. melo plantlets or plants resistant to Scab disease, aphids and Powdery Mildew (PM), which method comprises:
i. culturing in vitro an isolated cell or tissue of the C. melo plant according to the invention to produce C. melo micro-plantlets resistant to Scab disease, aphids and Powdery Mildew (PM), and
ii. optionally further subjecting the C. melo micro-plantlets to an in vivo culture phase to develop into C. melo plant resistant to Scab disease, aphids and Powdery Mildew (PM).

Further provided is a method for improving the yield of C. melo plants in an environment infested by Scab disease, aphids and Powdery mildew (PM), comprising growing C. melo plants resistant to Scab disease, aphids and PM and that optionally does not have any necrotic phenotype linked to the Zym gene, wherein said plant comprises in its genome (i) at least one QTL conferring resistance to Scab disease, wherein said at least one QTL is present on linkage group (LG) 2 and/or linkage group 5 (LG5), (ii) at least one QTL conferring resistance to PM, wherein said at least one QTL is present on LG2 and/or LG5, and is different from said at least one QTL in (i), and (iii) the Vat gene analog conferring resistance to aphids on LG5.

Also provided is a method for improving the yield of C. melo plants in an environment infested by Scab disease, aphids and Powdery mildew (PM) comprising:
a. identifying C. melo plants resistant to Scab disease, aphids and Powdery Mildew (PM) comprising in their genome (i) at least one QTL conferring resistance to Cladosporium, wherein said at least one QTL is present on linkage group (LG) 2 and/or linkage group 5 (LG5), (ii) at least one QTL conferring resistance to PM, wherein said at least one QTL is present on LG2 and/or LG5, and is different from said at least one QTL in (i), and (iii) the Vat gene analog conferring resistance to aphids on LG5, and
b. growing said resistant C. melo plants in said infested environment.

### Definitions

As used herein, the term “plant part” refers to any part of a plant including but not limited to the shoot, root, stem, seeds, fruits, leaves, petals, flowers, ovules, branches, petioles, internodes, pollen, stamen, rootstock, scion and the like.

As used herein, the term “Quantitative Trait Loci (QTL)” refers to a genomic region that may comprise one or more genes or regulatory sequences. A QTL may for instance comprise one or more genes of which products confer genetic resistance or tolerance. Alternatively, a QTL may for instance comprise regulatory genes or sequences of which products influence the expression of genes on other loci in the genome of the plant thereby conferring the resistance or tolerance. The QTLs of the present invention may be defined by indicating their genetic location in the genome of the respective pathogen-resistant accession using one or more molecular genomic markers. One or more markers, in turn, indicate a specific locus. Distances between loci are usually measured by frequency or crossing-over between loci on the same chromosome. The farther apart two are, the more likely that a crossover will occur between them. Conversely, if two loci are close together, a cross over is less likely to occur between them. As a rule, one centimorgan (cM) is equal to 1% recombination between loci (marker). When a QTL can be indicated by multiple markers, the genetic distance between the end-point markers is indicative of the size of the QTL.

The term “Resistance” is as defined by the ISF (International Seed Federation) Vegetable and Ornamental Crops Section for describing the reaction of plants to pests or pathogens, and abiotic stresses for the Vegetable Seed Industry.

Specifically, by resistance, it is meant the ability of a plant variety to restrict the growth and development of a specified pest or pathogen and/or the damage they cause when compared to susceptible plant varieties under similar environmental conditions and pest or pathogen pressure. Resistant varieties may exhibit some disease symptoms or damage under heavy pest or pathogen pressure.

By “tolerance” is meant the ability of a plant variety to endure biotic and abiotic stress without serious consequences for growth, appearance and yield.

As used herein, the term “susceptible” refers to a plant that is unable to restrict the growth and development of a specified pest or pathogen.

As used herein, the term “offspring” or “progeny” refers to any plant resulting as progeny from a vegetative or sexual reproduction from one or more parent plants or descendants thereof. For instance, an offspring plant may be obtained by cloning or selfing of a parent plant or by crossing two parents plants and include selfings as well as the F1 or F2 or still further generations. An F1 is a first-generation offspring produced from parents at least one of which is used for the first time as donor of a trait, while offspring of a second generation (F2) or subsequent generations (F3, F4, etc.) are specimens produced from selfing of F1's, F2s, etc. An F1 may thus be (and usually) a hybrid resulting from a cross between two true breeding parents (true-breeding is homozygous for a trait), while an F2 may be (and usually is) an offspring resulting from self-pollination of said F1 hybrids.

As used herein, the term “cross”, “crossing” refer to the process by which the pollen of one flower on one plant is applied (artificially or naturally) to the ovule (stigma) of a flower on another plant.

As used herein, the term “heterozygote” refers to a diploid or polyploidy cell or plant having different alleles (forms of a given gene or sequences) present at at least one locus.

As used herein, the term “heterozygous” refers to the presence of different alleles (forms of a given gene or sequences) at a particular locus.

As used herein, the term “homozygote” refers to an individual cell or plant having the same alleles at one or more loci on all homologous chromosomes.

As used herein, the term “homozygous” refers to the presence of identical alleles at one or more loci in homologous chromosomal segments.

As used herein, the term “hybrid” refers to any individual cell, tissue or plant resulting from a cross between parents that differ in one or more genes.

As used herein, the term “inbred” or “line” refers to a relatively true-breeding strain.

As used herein, the term “phenotype” refers to the observable characters of an individual cell, cell culture, organism (e.g. a plant), or group of organisms which results from the interaction between that individual genetic makeup (i.e. genotype) and the environment.

As used herein, the terms “introgression”, “introgressed” and “introgressing” refer to the process whereby genes of one species, variety or cultivar are moved into the genome of another species, variety or cultivar, by crossing those species. The crossing may be natural or artificial. The process may be optionally be completed by backcrossing to the recurrent parent, in which case introgression refers to infiltration of the genes of one species into the gene pool of another through repeated backcrossing of an interspecific hybrid with one of its parents. An introgression may be also described as a heterologous genetic material stably integrated in the genome of a recipient plant.

As used herein, the terms “molecular marker” refer to an indicator that is used in methods for visualizing differences in characteristics of nucleic acid sequences. Examples of such indicators are restriction fragment length polymorphism (RFLP) markers, amplification fragment length polymorphism (AFLP) markers, single nucleotide polymorphisms (SNPs), insertion mutations, microsatellite markers (SSRs), sequence-characterized amplified regions (SCARs), cleaved amplified polymorphic sequence (CAPS) markers or isozyme markers or combinations of the markers described herein which defines a specific genetic and chromosomal location. Mapping of molecular markers in the vicinity of an allele is a procedure which can be performed quite easily by the person skilled in the art using common molecular techniques.

As used herein, the term “primer” refers to an oligonucleotide which is capable of annealing to the amplification target allowing a DNA polymerase to attach, thereby serving as a point of initiation of DNA synthesis when placed under conditions in which synthesis of primers extension product is induced, i.e., in the presence of nucleotides and an agent for polymerization such as DNA polymerase and at a suitable temperature and pH. The primer is preferably single stranded for maximum efficiency in amplification. Preferably, the primer is an oligodeoxyribonucleotide. The primer must be sufficiently long to prime the synthesis of extension products in the presence of the agent for polymerization. The exact length of the primers will depend on many factors, including temperature and composition (A/T and G/C content) of primer. A pair of bi-directional primers consists of one forward and one reverse primer as commonly used in the art of DNA amplification such as in PCR amplification.

By “Scab disease”, it is meant a fungal disease that is caused by fungi of the Ascomycota class (indoor and outdoor molds) named Cladosporium. Non-limiting examples of Cladosporium species include Cladosporium elegans, Cladosporium cladosporioides, Cladosporium caryigenum, Cladosporium musae, Cladosporium brassicae, Cladosporium cucumerinum and Cladosporium oxysporum. Preferably, the Cladosporium species is the Cladosporium cucumerinum (also known as Cladosporium cucumerinum Ellis and Arthur). The disease symptoms can be present on all parts of the plant, including leaves, petioles, stem and fruit. Leaf lesions begin as pale green, water-soaked areas that gradually turn gray to white and become angular. Often, lesions are surrounded by a yellowish halo, and the lesion center tears away, leaving ragged holes in the leaves. On fruit, small (⅛-inch), gray, slightly sunken, oozing, gummy spots develop that resemble insect “stings”. Later, the spots enlarge and finally become distinct sunken cavities. The crater-like depressions develop an irregular, scab-like appearance as fruit age, and might ooze a gummy substance (https://extension.illinois.edu/hortanswers/detailproblem.cfm?PathogenID=141).

By “Powdery mildew”, it is meant a fungal disease that is caused by fungi of the order Erisyphales. Preferably, powdery mildew is caused by Golovinomyces cichoracearum (also known as Erysiphe cichoracearum DC) and/or Podosphaera xanthii (also known as Sphaerotheca fuliginea or Oidium erysiphoides). Still preferably, the powdery mildew is caused by Podosphaera xanthii. In some embodiments, the powdery mildew is caused by the Podosphaera xanthii races Px-1, Px-2, Px-3, Px-5, and/or Px3-5. The disease is characterized by white or pale yellow lesions on stems, petioles, upper and/or lower leaf surface, and the fruit. When the disease progress, the lesions get larger and denser. As the lesions enlarge, conidia are produced from affected tissue and the spots take on a powdery appearance. Such sporulation of the fungus may cause a cell destruction of the plant tissues, and the loss of the photosynthesis efficacy leading to a less energy performance of the plant (https://cuccap.org/disease-management/melon/powdery-mildew/).

By “aphid”, it is meant an insect pest that colonizes a wide range of economically important host plants, such as cucurbits, for which it is the major pest. Colonization of cucurbits by aphids causes stunting and severe leaf curling that can result in plant death. Aphids are also efficient virus vectors and thus contribute to the spreading of viral diseases. Preferably, the aphid is of the species Aphis gossypii Glover.

By “Vat gene analog”, it is meant the major gene responsible for the resistance to aphids, and more preferably to Aphis gossypii Glover, of the PI 414723 accession as described in Dogimont et al., 2008, Pitrat M. (ed), Cucurbitaceae 2008, Proceedings of the IXth EUCARPIA meeting on genetics and breeding of Cucurbitaceae, Avignon (France), May 21-24th, 2008, pp. 219-228. The vat gene analog is located on LG5 (Dogimont et al., 2014, The Plant Journal, 80, 993-1004). In some embodiments, the Vat gene analog comprises or consists of a nucleotidic sequence of 5897 bp having 99.8% of identity with the nucleic sequence encoded by the vat gene referenced under the GenBank Number KM513660.1, update Mar. 24, 2015 (SEQ ID NO: 46). In some embodiments, the vat gene analog encodes a polypeptide having an amino-acid sequence of 1473 aa that has 99.6% of identity with the vat protein referenced under the GenBank Number AIU36098.1, update Oct. 27, 2014 (SEQ ID NO: 47), i.e the Vat gene analog encodes a polypeptide which has 6 different aa relative to the vat protein referenced under the GenBank Number AIU36098.1, update Oct. 27, 2014 (SEQ ID NO: 47). Identification of the Vat gene analog may be performed as described in the patent application FR 2 849 863 using the forward primer Me-VatE-F having the sequence 5′-CTCCACTCAGAATTGGTAGGTGCC-3′ (SEQ ID NO: 48) and the reverse primer Me-VatE-R having the sequence 5′-CCTTAGAAGAAGATGAAGTCTCCC-3′ (SEQ ID NO: 49). Using the above-mentioned pair of primers, detection of a fragment of 1723 bp indicates the presence of the Vat gene analog.

In the context of the present application, the percentage of identity is calculated using a global alignment (i.e. the two sequences are compared over their entire length). Methods for comparing the identity of two or more sequences are well known in the art. The «needle» program, which uses the Needleman-Wunsch global alignment algorithm (Needleman and Wunsch, 1970 J. Mol. Biol. 48:443-453) to find the optimum alignment (including gaps) of two sequences when considering their entire length, may for example be used. The needle program is for example available on the ebi.ac.uk World Wide Web site. The percentage of identity in accordance with the invention is preferably calculated using the EMBOSS::needle (global) program with a “Gap Open” parameter equal to 10.0, a “Gap Extend” parameter equal to 0.5, and a Blosum62 matrix.

In the context of the invention, DNA strand and allele designation and orientation for the markers LG2-M1, Cm_MU47536_461, LG2-M2, Cm_MU47380_465, LG2-M3, Cm_MU45136_209, LG2-M4, Cm_MU45398_32, Cm_MU46579_322, Cm_MU44050_58, Cm_MU45437_855 and LG5-M3 can be done according to the TOP/BOT method developed by Illumina (https://www.illumina.com/documents/products/technotes/technote_topbot.pdf).

The Cm suffix in the SNP name can be omitted in the following, however referring to the same markers of Cucumis melo.

In the context of the present application, a chromosomal region delimited by two markers X and Y (e.g. SNPs) refers to the section of the chromosome lying between the positions of these two markers and comprising said markers, therefore the nucleotide sequence of this chromosomal region begins with the nucleotide corresponding to marker X and ends with the nucleotide corresponding to marker Y, i.e. the markers are comprised within the region they delimit.

By “necrotic phenotype linked to the Zym gene”, it is meant the necrotic phenotype as described in Pitrat and Lecoq, 1984, Euphytica, 33(1):57-61 in presence of the Zym gene homozygously. More specifically, such a necrotic phenotype corresponds to the appearance of necrotic spots in the epidermis of the leaf which extent gradually on the leaf and can lead to completely drying the leaf or of the stem.

By “commercially acceptable fruit quality”, it is meant a fruit of type Charentais, Western Shipper, Harper or Italian Cantaloup with a flesh color going from the orange to the red Magenta (i.e. a colorimetry having L*c*h values: 60<L<65, 40<c<50 and 66<h75 as measured by a Minolta colorimeter), a flesh firmness with a mean value of 4 kg/0.5 cm²+/−2 as measured by a penefel, a level of Brix of at least 11°, a diameter of the fruit/size of the cavity ratio as found as in the Vedrantais variety, and a fruit preservation greater than 8 days at +12° C. Examples of fruits having a commercially acceptable fruit quality can be the fruits of the HUGO, ALONSO or FELINO varieties from HM-CLAUSE.

By association, or genetic association, and more specifically genetic linkage, it is to be understood that a polymorphism of a genetic marker (e.g. a specific allele of the SNP marker) and the phenotype of interest occur simultaneously, i.e. are inherited together, more often than would be expected by chance occurrence, i.e. there is a non-random association of the allele and of the genetic sequences responsible for the phenotype, as a result of their genomic proximity.

### SEQUENCE LISTING

SEQ ID NO: 1 shows a flanking sequence of the marker Cm_MU45136_209.

SEQ ID NO: 2 shows the sequence of a forward primer for detecting the susceptible allele of Cm_MU45136_209 marker.

SEQ ID NO: 3 shows the sequence of a forward primer for detecting the resistant allele of Cm_MU45136_209 marker.

SEQ ID NO: 4 shows the sequence of a common reverse primer for Cm_MU45136_209 marker detection.

SEQ ID NO: 5 shows a flanking sequence of the marker LG2-M4.

SEQ ID NO: 6 shows the sequence of a forward primer for detecting the susceptible allele of LG2-M4 marker.

SEQ ID NO: 7 shows the sequence of a forward primer for detecting the resistant allele of LG2-M4 marker.

SEQ ID NO: 8 shows the sequence of a common reverse primer for LG2-M4 marker detection.

SEQ ID NO: 9 shows a flanking sequence of the marker Cm_MU45398_32.

SEQ ID NO: 10 shows the sequence of a forward primer for detecting the susceptible allele of Cm_MU45398_32 marker.

SEQ ID NO: 11 shows the sequence of a forward primer for detecting the resistant allele of Cm_MU45398_32 marker.

SEQ ID NO: 12 shows the sequence of a common reverse primer for Cm_MU45398_32 marker detection.

SEQ ID NO: 13 shows a flanking sequence of the marker LG5-M1.

SEQ ID NO: 14 shows the sequence of a forward primer for CMCTN2 marker detection.

SEQ ID NO: 15 shows the sequence of a reverse primer for CMCTN2 marker detection.

SEQ ID NO: 16 shows a flanking sequence of the marker Cm_MU46579_322.

SEQ ID NO: 17 shows the sequence of a forward primer for detecting the susceptible allele of Cm_MU46579_322 marker.

SEQ ID NO: 18 shows the sequence of a forward primer for detecting the resistant allele of Cm_MU46579_322 marker.

SEQ ID NO: 19 shows the sequence of a common reverse primer for Cm_MU46579_322 marker detection.

SEQ ID NO: 20 shows a flanking sequence of the marker Cm_MU44050_58.

SEQ ID NO: 21 shows the sequence of a forward primer for detecting the susceptible allele of Cm_MU44050_58 marker.

SEQ ID NO: 22 shows the sequence of a forward primer for detecting the resistant allele of Cm_MU44050_58 marker.

SEQ ID NO: 23 shows the sequence of a common reverse primer for Cm_MU44050_58 marker detection.

SEQ ID NO: 24 shows the sequence of a forward primer for CMBR120 marker detection.

SEQ ID NO: 25 shows the sequence of a reverse primer for CMBR120 marker detection.

SEQ ID NO: 26 shows a flanking sequence of the marker LG2-M1.

SEQ ID NO: 27 shows the sequence of a forward primer for detecting the susceptible allele of LG2-M1 marker.

SEQ ID NO: 28 shows the sequence of a forward primer for detecting the resistant allele of LG2-M1 marker.

SEQ ID NO: 29 shows the sequence of a common reverse primer for LG2-M1 marker detection.

SEQ ID NO: 30 shows a flanking sequence of the marker Cm_MU47536_461.

SEQ ID NO: 31 shows the sequence of a forward primer for detecting the susceptible allele of Cm_MU47536_461 marker.

SEQ ID NO: 32 shows the sequence of a forward primer for detecting the resistant allele of Cm_MU47536_461 marker.

SEQ ID NO: 33 shows the sequence of a common reverse primer for Cm_MU47536_461 marker detection.

SEQ ID NO: 34 shows a flanking sequence of the marker Cm_MU45437_855.

SEQ ID NO: 35 shows the sequence of a forward primer for detecting the susceptible allele of Cm_MU45437_855 marker.

SEQ ID NO: 36 shows the sequence of a forward primer for detecting the resistant allele of Cm_MU45437_855 marker.

SEQ ID NO: 37 shows the sequence of a common reverse primer for Cm_MU45437_855 marker detection.

SEQ ID NO: 38 shows the sequence of a forward primer for LG5-M2 marker detection.

SEQ ID NO: 39 shows the sequence of a reverse primer for LG5-M2 marker detection.

SEQ ID NO: 40 shows the sequence of a forward primer for CMTAN139 marker detection.

SEQ ID NO: 41 shows the sequence of a reverse primer for CMTAN139 marker detection.

SEQ ID NO: 42 shows a flanking sequence of the marker LG5-M3.

SEQ ID NO: 43 shows the sequence of a forward primer for detecting the susceptible allele of LG5-M3 marker.

SEQ ID NO: 44 shows the sequence of a forward primer for detecting the resistant allele of LG5-M3 marker.

SEQ ID NO: 45 shows the sequence of a common reverse primer for LG5-M3 marker detection.

SEQ ID NO: 46 represents the genomic sequence of the Vat gene referenced under the GenBank Number KM513660.1.

SEQ ID NO: 47 represents the amino acid sequence of the Vat protein referenced under the GenBank Number AIU36098.1.

SEQ ID NO: 48 shows the sequence of a forward primer for amplifying the Vat gene analog.

SEQ ID NO: 49 shows the sequence of a reverse primer for amplifying the Vat gene analog.

SEQ ID NO: 50 shows a flanking sequence of the marker LG2-M2.

SEQ ID NO: 51 shows the sequence of a forward primer for detecting the susceptible allele of LG2-M2 marker.

SEQ ID NO: 52 shows the sequence of a forward primer for detecting the resistant allele of LG2-M2 marker.

SEQ ID NO: 53 shows the sequence of a common reverse primer for LG2-M2 marker detection.

SEQ ID NO: 54 shows a flanking sequence of the marker Cm_MU47380_465.

SEQ ID NO: 55 shows the sequence of a forward primer for detecting the susceptible allele of Cm_MU47380_465 marker.

SEQ ID NO: 56 shows the sequence of a forward primer for detecting the resistant allele of Cm_MU47380_465 marker.

SEQ ID NO: 57 shows the sequence of a common reverse primer for Cm_MU47380_465 marker detection.

SEQ ID NO: 58 shows a flanking sequence of the marker LG2-M3.

SEQ ID NO: 59 shows the sequence of a forward primer for detecting the susceptible allele of LG2-M3 marker.

SEQ ID NO: 60 shows the sequence of a forward primer for detecting the resistant allele of LG2-M3 marker.

SEQ ID NO: 61 shows the sequence of a common reverse primer for LG2-M3 marker detection.

### LEGEND OF THE FIGURES

FIG. 1: this figure comprises pictures illustrating the different melon lines and accessions used in the examples—one photograph of the exterior aspect, and one photograph of the interior aspect.

FIG. 2: this figure is a representation of a genetic map of LG2, as disclosed in Diaz et al, 2011, on which the markers of the present invention have been added, as well as the corresponding QTL. PM stands for Powdery Mildew resistance; Scab for resistance to Scab disease, and ZYMV for resistance to Zucchini Yellow Mosaic Virus.

FIG. 3: this figure is a representation of a genetic map of LG5, as disclosed in Diaz et al, 2011, on which the markers of the present invention have been added, as well as the corresponding QTL. PM stands for Powdery Mildew resistance; Scab for resistance to Scab disease.

### DETAILED DESCRIPTION OF THE INVENTION

According to a first embodiment, the present invention is thus directed to a Cucumis melo (C. melo) plant that is resistant to Scab disease, aphids and Powdery Mildew (PM), wherein said plant:
comprises:
   (i) at least one QTL conferring resistance to Scab disease, wherein said at least one QTL is present on linkage group 2 (LG2) and/or linkage group 5 (LG5),
   (ii) at least one QTL conferring resistance to PM, wherein at least one QTL is present on LG2 and/or LG5, and is different from said one or more QTL in (i), and
   (iii) the vat gene analog associated to aphids resistance on LG5, and
has a commercially acceptable fruit quality.

According to a preferred embodiment, the plant does not have any necrotic phenotype linked to the Zym gene.

By “one or more QTL conferring resistance to Scab disease”, it has to be understood at least 1, 2, 3, 4, 5 or more QTLs conferring resistance to Scab disease, i.e. at least 1, 2, 3, 4, 5 or more QTLs that is/are present on LG2 and/or at least 1, 2, 3, 4, 5 or more QTLs is/are present on LG5.

By “one or more QTL conferring resistance to PM”, it has to be understood at least 1, 2, 3, 4, 5 or more QTLs conferring resistance to PM, i.e. at least 1, 2, 3, 4, 5 or more QTLs that is/are present on LG2 and/or at least 1, 2, 3, 4, 5 or more QTLs that is/are present on LG5, said QTLs conferring resistance to PM on LG2 and/or LG5 being different from the QTLs conferring resistance to Scab disease.

In some embodiments, said QTL conferring resistance to Scab disease that is present on LG2 is located within a chromosomal region that is delimited by marker Cm_MU45136_209 and marker Cm_MU45398_32.

In some embodiments, said QTL conferring resistance to Scab disease that is present on LG5 is located within a chromosomal region that is delimited by marker LG5-M1 and marker Cm_MU44050_58.

In some embodiments, said QTL conferring resistance to PM that is present on LG2 is located within a chromosomal region that is delimited by marker CMBR120 and marker Cm_MU47536_461.

In some embodiments, said QTL conferring resistance to PM that is present on LG5 is located within a chromosomal region that is delimited by marker Cm_MU45437_855 and marker LG5-M3.

Preferably, the C. melo plant according to the invention comprises any combination of the QTLs as defined hereabove, has a commercially acceptable fruit quality and does not have any necrotic phenotype. For example, the C. melo plant according to the invention may comprises the following combination of the QTL(s) associated to resistance to Scab disease, Aphids, and PM:
(a) a QTL conferring resistance to Scab disease that is located on LG2 within a chromosomal region delimited by marker Cm_MU45136_209 and marker Cm_MU45398_32, a QTL conferring resistance to PM that is located on LG2 within a chromosomal region delimited by marker CMBR120 and marker Cm_MU47536_461, and the Vat gene analog associated to aphids resistance on LG5;
(b) a QTL conferring resistance to Scab disease that is located on LG5 within a chromosomal region delimited by marker LG5-M1 and marker Cm_MU44050_58, a QTL conferring resistance to PM that is located on LG5 within a chromosomal region delimited by marker Cm_MU45437_855 and marker LG5-M3, and the Vat gene associated to aphids resistance on LG5;
(c) a QTL conferring resistance to Scab disease that is located on LG2 within a chromosomal region delimited by marker Cm_MU45136_209 and marker Cm_MU45398_32, a QTL conferring resistance to PM that is located on LG5 within a chromosomal region delimited by marker Cm_MU45437_855 and marker LG5-M3, and the Vat gene analog associated to aphids resistance on LG5;
(d) a QTL conferring resistance to Scab disease that is located on LG5 within a chromosomal region delimited by marker LG5-M1 and marker Cm_MU44050_58, a QTL conferring resistance to PM that is located on LG2 within a chromosomal region delimited by marker CMBR120 and marker Cm_MU47536_461, and the Vat gene analog associated to aphids resistance on LG5;
(e) a QTL conferring resistance to Scab disease that is located on LG2 within a chromosomal region delimited by marker Cm_MU45136_209 and marker Cm_MU45398_32, a QTL conferring resistance to Scab disease that is located on LG5 within a chromosomal region delimited by marker LG5-M1 and marker Cm_MU44050_58, a QTL conferring resistance to PM that is located on LG2 within a chromosomal region delimited by marker CMBR120 and marker Cm_MU47536_461, and the Vat gene analog associated to aphids resistance on LG5;
(f) a QTL conferring resistance to Scab disease that is located on LG2 within a chromosomal region delimited by marker Cm_MU45136_209 and marker Cm_MU45398_32, a QTL conferring resistance to Scab disease that is located on LG5 within a chromosomal region delimited by marker LG5-M1 and marker Cm_MU44050_58, a QTL conferring resistance to PM that is located on LG5 within a chromosomal region delimited by marker Cm_MU45437_855 and marker LG5-M3, and the Vat gene analog associated to aphids resistance on LG5;
(g) a QTL conferring resistance to Scab disease that is located on LG2 within a chromosomal region delimited by marker Cm_MU45136_209 and marker Cm_MU45398_32, a QTL conferring resistance to PM that is located on LG2 within a chromosomal region delimited by marker CMBR120 and marker Cm_MU47536_461, a QTL conferring resistance to PM that is located on LG5 within a chromosomal region delimited by marker Cm_MU45437_855 and marker LG5-M3, and the Vat gene analog associated to aphids resistance on LG5;
(h) a QTL conferring resistance to Scab disease that is located on LG5 within a chromosomal region delimited by marker LG5-M1 and marker Cm_MU44050_58, a QTL conferring resistance to PM that is located on LG2 within a chromosomal region delimited by marker CMBR120 and marker Cm_MU47536_461, a QTL conferring resistance to PM that is located on LG5 within a chromosomal region delimited by marker Cm_MU45437_855 and marker LG5-M3, and the Vat gene analog associated to aphids resistance on LG5; or
(i) a QTL conferring resistance to Scab disease that is located on LG2 within a chromosomal region delimited by marker Cm_MU45136_209 and marker Cm_MU45398_32, a QTL conferring resistance to Scab disease that is located on LG5 within a chromosomal region delimited by marker LG5-M1 and marker Cm_MU44050_58, a QTL conferring resistance to PM that is located on LG2 within a chromosomal region delimited by marker CMBR120 and marker Cm_MU47536_461, a QTL conferring resistance to PM that is located on LG5 within a chromosomal region delimited by marker Cm_MU45437_855 and marker LG5-M3, and the Vat gene analog associated to aphids resistance on LG5.

Thus in a particular preferred embodiment, the C. melo plant according to the invention:
comprises a QTL conferring resistance to Scab disease that is located on LG2 within a chromosomal region that is delimited by marker Cm_MU45136_209 and marker Cm_MU45398_32, a QTL conferring resistance to Scab disease that is located on LG5 within a chromosomal region delimited by marker LG5-M1 and marker Cm_MU44050_58, a QTL conferring resistance to PM that is located on LG2 within a chromosomal region that is delimited by marker CMBR120 and marker Cm_MU47536_461, a QTL conferring resistance to PM that is located on LG5 within a chromosomal region that is delimited by marker Cm_MU45437_855 and marker LG5-M3, and the Vat gene analog associated to aphids resistance on LG5,
has a commercially acceptable fruit quality, and
does not have any necrotic phenotype linked to the Zym gene.

In some embodiments, said QTL on LG2 conferring resistance to Scab disease is identified by Cm_MU45136_209, LG2-M4 (SEQ ID NO: 5), and/or Cm_MU45398_32 markers detection; or any other markers within the chromosomal region delimited by marker Cm_MU45136_209 and marker Cm_MU45398_32. In some embodiments, detection of the Cm_MU45136_209, LG2-M4, and/or Cm_MU45398_32 markers is performed by amplification, preferably by PCR, using specific primers which can be used to amplify the resistant/susceptible allele of each of the Cm_MU45136_209, LG2-M4, and Cm_MU45398_32 markers.

In particular, detection of the Cm_MU45136_209 marker on LG2 is performed using two forward primers, one being specific for the resistant allele and one being specific for the susceptible allele, and one common reverse primer. Said two forward primers may be selected so as to enable amplifying a nucleic acid comprising or consisting of SEQ ID NO: 1, or a fragment thereof including the [T/C] polymorphism at position 209 of SEQ ID NO: 1. For example, the forward primer for detecting the susceptible allele of the marker Cm_MU45136_209, by amplifying a nucleic acid consisting of sequence SEQ ID NO: 1 or its complementary sequence, may consist of the sequence 5′-ACAAATTTCTTGGAGCTGCAAGACTTA-3′ (SEQ ID NO: 2), the forward primer for detecting the resistant allele of the marker Cm_MU45136_209, by amplifying a nucleic acid consisting of sequence SEQ ID NO: 1 or its complementary sequence, may consist of the sequence 5′-CAAATTTCTTGGAGCTGCAAGACTTG-3′ (SEQ ID NO: 3), and the common reverse primer may consist of the sequence 5′-TATCATCGGTTCTTGTCTCAAGAAGGAAA-3′ (SEQ ID NO: 4). Using primers consisting of sequences SEQ ID NO: 2, SEQ ID NO: 3 and SEQ ID NO: 4, detection of a guanine (G) rather than an adenine (A), or a cytosine (C) rather than a thymine (T) in the complementary strand, at position 209 of the amplification product consisting of sequence SEQ ID NO: 1 indicates the presence of the QTL on LG2 conferring resistance to Scab disease (see Table 1 below).

In particular, detection of the LG2-M4 marker on LG2 is performed using two forward primers, one being specific for the resistant allele and one being specific for the susceptible allele, and one common reverse primer. Said two forward primers may be selected so as to enable amplifying a nucleic acid comprising or consisting of SEQ ID NO: 5, or a fragment thereof including the [G/A] polymorphism at position 61 of SEQ ID NO: 5. For example, the forward primer for detecting the susceptible allele of the marker LG2-M4, by amplifying a nucleic acid consisting of sequence SEQ ID NO: 5, may consist of the sequence 5′-TTCACACCATTTGTAAGTTTGAACTTTG-3′ (SEQ ID NO: 6), the forward primer for detecting the resistant allele of the marker LG2-M4, by amplifying a nucleic acid consisting of sequence SEQ ID NO: 5, may consist of the sequence 5′-GTTTTCACACCATTTGTAAGTTTGAACTTTA-3′ (SEQ ID NO: 7), and the common reverse primer may consist of the sequence 5′-GCACGTATGATAACGAGTTCTTTAGTGTT-3′ (SEQ ID NO: 8). Using primers consisting of sequences SEQ ID NO: 6, SEQ ID NO: 7 and SEQ ID NO: 8, detection of an adenine (A) rather than a guanine (G) at position 61 of the amplification product consisting of sequence SEQ ID NO: 5 indicates the presence of the QTL on LG2 conferring resistance to Scab disease (see Table 1 below).

In particular, detection of the Cm_MU45398_32 marker on LG2 is performed using two forward primers, one being specific for the resistant allele and one being specific for the susceptible allele, and one common reverse primer. Said two forward primers may be selected so as to enable amplifying a nucleic acid comprising or consisting of SEQ ID NO: 9, or a fragment thereof including the [T/C] polymorphism at position 32 of SEQ ID NO: 9. For example, the forward primer for detecting the susceptible allele of the marker Cm_MU45398_32, by amplifying a nucleic acid consisting of sequence SEQ ID NO: 9, may consist of the sequence 5′-CAAAACAGGGTTGTTCCGCTTTACT-3′ (SEQ ID NO: 10), the forward primer for detecting the resistant allele of the marker Cm_MU45398_32, by amplifying a nucleic acid consisting of sequence SEQ ID NO: 9, may consist of the sequence 5′-AAAACAGGGTTGTTCCGCTTTACC-3′ (SEQ ID NO: 11), and the common reverse primer may consist of the sequence 5′-CGTCTTCTTCTTCTTCTTCTTTGTTGCTA-3′ (SEQ ID NO: 12). Using primers consisting of sequences SEQ ID NO: 10, SEQ ID NO: 11 and SEQ ID NO: 12, detection of a cytosine (C) rather than a thymine (T) at position 32 of the amplification product consisting of sequence SEQ ID NO: 9 indicates the presence of the QTL on LG2 conferring resistance to Scab disease (see Table 1 below).

In some embodiments, said QTL on LG5 conferring resistance to Scab disease is identified by LG5-M1, CMCTN2, Cm_MU46579_322, and/or Cm_MU44050_58 markers detection; or any other markers within the chromosomal region delimited by marker LG5-M1 and marker Cm_MU44050_58. In some embodiments, detection of the LG5-M1, CMCTN2, Cm_MU46579_322, and/or Cm_MU44050_58 markers is performed by amplification, preferably by PCR, using specific primers which can be used to amplify the resistant/susceptible allele of each of the LG5-M1, CMCTN2, Cm_MU46579_322, and/or Cm_MU44050_58 markers.

In particular, detection of the LG5-M1 marker on LG5 is performed using two forward primers, one being specific for the resistant allele and one being specific for the susceptible allele, and one common reverse primer. Said two forward primers may be selected so as to enable amplifying a nucleic acid comprising or consisting of SEQ ID NO: 13, or a fragment thereof including the [T/C] polymorphism at position 36 of SEQ ID NO: 13 (see Table 1 below). Adequate primers which can be used for example in a KASPar assay can easily be designed by a skilled person. Using primers allowing to detect the [T/C] polymorphism at position 36 of SEQ ID NO: 13, detection of a guanine (G) rather than an adenosine (A), or a cytosine (C) rather than a thymine (T) in the complementary strand, at position 36 of the amplification product consisting of sequence SEQ ID NO: 13 indicates the presence of the QTL on LG2 conferring resistance to Scab disease (see Table 1 below).

In particular, detection of the CMCTN2 marker on LG5 is performed by PCR using a forward primer and a reverse primer which can be used to amplify the resistant/susceptible allele of the CMCTN2 marker. In some embodiments, said PCR is followed by digestion of the amplification products with restriction enzyme or by sequencing the amplification product. In particular, the forward primer and the reverse primer for amplifying the CMCTN2 marker may respectively comprise the sequences 5′-CTGAAAGCAGTTTGTGTCGA-3′ (SEQ ID NO: 14) and 5′-AAAGAAGGAAGAGGCTGAGA-3′ (SEQ ID NO: 15). Using primers consisting of SEQ ID NO: 14 and SEQ ID NO: 15, detection of an amplification product of 195 bp indicates presence of the QTL on LG5 conferring resistance to Scab disease (see Table 2 below).

In particular, detection of the Cm_MU46579_322 marker on LG5 is performed using two forward primers, one being specific for the resistant allele and one being specific for the susceptible allele, and one common reverse primer. Said two forward primers may be selected so as to enable amplifying a nucleic acid comprising or consisting of SEQ ID NO: 16, or a fragment thereof including the [T/C] polymorphism at position 51 of SEQ ID NO: 16. For example, the forward primer for detecting the susceptible allele of the marker Cm_MU46579_322, by amplifying a nucleic acid consisting of sequence SEQ ID NO: 16, may consist of the sequence 5′-TCCGATCCTCACTGGAACTATCT-3′ (SEQ ID NO: 17), the forward primer for detecting the resistant allele of the marker Cm_MU46579_322, by amplifying a nucleic acid consisting of sequence SEQ ID NO: 16, may consist of the sequence 5′-CCGATCCTCACTGGAACTATCC-3′ (SEQ ID NO: 18), and the common reverse primer may consist of the sequence 5′-CAGCCTCATCGACTGTGAACTTCAT-3′ (SEQ ID NO: 19). Using primers consisting of sequences SEQ ID NO: 17, SEQ ID NO: 18 and SEQ ID NO: 19, detection of a guanine (G) rather than an adenine (A), or a cytosine (C) rather than a thymine (T) in the complementary strand, at position 51 of the amplification product consisting of sequence SEQ ID NO: 16 indicates the presence of the QTL on LG5 conferring resistance to Scab disease (see Table 1 below).

In particular, detection of the Cm_MU44050_58 marker on LG5 is performed using two forward primers, one being specific for the resistant allele and one being specific for the susceptible allele, and one common reverse primer. Said two forward primers may be selected so as to enable amplifying a nucleic acid comprising or consisting of SEQ ID NO: 20, or a fragment thereof including the [T/C] polymorphism at position 58 of SEQ ID NO: 20. For example, the forward primer for detecting the susceptible allele of the marker Cm_MU44050_58, by amplifying a nucleic acid consisting of sequence SEQ ID NO: 20, may consist of the sequence 5′-GCGTTGCTTTCATGGCGAGCTTT-3′ (SEQ ID NO: 21), the forward primer for detecting the resistant allele of the marker Cm_MU44050_58, by amplifying a nucleic acid consisting of sequence SEQ ID NO: 20, may consist of the sequence 5′-CGTTGCTTTCATGGCGAGCTTC-3′ (SEQ ID NO: 22), and the common reverse primer may consist of the sequence 5′-CTGTGGAACGGAGAAGCTCAAAGAA-3′ (SEQ ID NO: 23). Using primers consisting of sequences SEQ ID NO: 21, SEQ ID NO: 22 and SEQ ID NO: 23, detection of a guanine (G) rather than an adenine (A), or a cytosine (C) rather than a thymine (T) in the complementary strand, at position 58 of the amplification product consisting of sequence SEQ ID NO: 20 indicates the presence of the QTL on LG5 conferring resistance to Scab disease (see Table 1 below).

In some embodiments, said QTL on LG2 conferring resistance to PM is identified by CMBR120, LG2-M1 (SEQ ID NO: 26), and/or Cm_MU47536_461 markers detection; or any other markers within the chromosomal region delimited by marker CMBR120 and marker Cm_MU47536_461. In some embodiments, detection of the CMBR120, LG2-M1, and/or Cm_MU47536_461 markers is performed by amplification, preferably by PCR, using specific primers which can be used to amplify the resistant/susceptible allele of each of the CMBR120, LG2-M1, and/or Cm_MU47536_461 markers.

In particular, detection of the CMBR120 marker on LG2 is performed by PCR using a forward primer and a reverse primer which can be used to amplify the resistant/susceptible allele of the CMBR120 marker. In some embodiments, said PCR is followed by digestion of the amplification products with restriction enzyme or by sequencing the amplification product. In particular, the forward primer and the reverse primer for amplifying the CMBR120 marker may respectively comprise the sequences 5′-CTGGCCCCCTCCTAAACTAA-3′ (SEQ ID NO: 24) and 5′-CAAAAAGCATCAAAATGGTTG-3′ (SEQ ID NO: 25). Using primers consisting of SEQ ID NO: 24 and SEQ ID NO: 25, detection of an amplification product of 165 bp indicates presence of the QTL on LG2 conferring resistance to PM (see Table 2 below).

In particular, detection of the LG2-M1 marker on LG2 is performed using two forward primers, one being specific for the resistant allele and one being specific for the susceptible allele, and one common reverse primer. Said two forward primers may be selected so as to enable amplifying a nucleic acid comprising or consisting of SEQ ID NO: 26, or a fragment thereof including the [C/T] polymorphism at position 69 of SEQ ID NO: 26. For example, the forward primer for detecting the susceptible allele of the marker LG2-M1, by amplifying a nucleic acid consisting of sequence SEQ ID NO: 26, or its complementary sequence, may consist of the sequence 5′-CCTCATTTGGGCCCCGGG-3′ (SEQ ID NO: 27), the forward primer for detecting the resistant allele of the marker LG2-M1, by amplifying a nucleic acid consisting of sequence SEQ ID NO: 26, or its complementary sequence, may consist of the sequence 5′-AATCCTCATTTGGGCCCCGGA-3′ (SEQ ID NO: 28), and the common reverse primer may consist of the sequence 5′-TCATGGCTTCTGATACTCGTTCTGATAT-3′ (SEQ ID NO: 29). Using primers consisting of sequences SEQ ID NO: 27, SEQ ID NO: 28 and SEQ ID NO: 29, detection of an adenine (A) rather than a guanine (G), or a thymine (T) rather than a cytosine (C) in the complementary strand at position 69 of the amplification product consisting of sequence SEQ ID NO: 26 indicates the presence of the QTL on LG2 conferring resistance to PM (see Table 1 below).

In particular, detection of the Cm_MU47536_461 marker on LG2 is performed using two forward primers, one being specific for the resistant allele and one being specific for the susceptible allele, and one common reverse primer. Said two forward primers may be selected so as to enable amplifying a nucleic acid comprising or consisting of SEQ ID NO: 30, or a fragment thereof including the [A/T] polymorphism at position 51 of SEQ ID NO: 30. For example, the forward primer for detecting the susceptible allele of the marker Cm_MU47536_461, by amplifying a nucleic acid consisting of sequence SEQ ID NO: 30, may consist of the sequence 5′-ATGTACAAGATTTTGATAATGTGATTGATACA-3′ (SEQ ID NO: 31), the forward primer for detecting the resistant allele of the marker Cm_MU47536_461, by amplifying a nucleic acid consisting of sequence SEQ ID NO: 30, may consist of the sequence 5′-ATGTACAAGATTTTGATAATGTGATTGATACT-3′ (SEQ ID NO: 32), and the common reverse primer may consist of the sequence 5′-CGAAGAATATTAGCTGAGCCTTTGATGTT-3′ (SEQ ID NO: 33). Using primers consisting of sequences SEQ ID NO: 31, SEQ ID NO: 32 and SEQ ID NO: 33, detection of a thymine (T) rather than an adenine (A) at position 51 of the amplification product consisting of sequence SEQ ID NO: 30 indicates the presence of the QTL on LG2 conferring resistance to PM (see Table 1 below).

In some embodiments, said QTL on LG5 conferring resistance to PM is identified by Cm_MU45437_855, LG5-M2, CMTAN139, and/or LG5-M3 markers detection; or any other markers within the chromosomal region delimited by marker Cm_MU45437_855 and marker LG5-M3. In some embodiments, detection of the Cm_MU45437_855, LG5-M2, CMTAN139, and/or LG5-M3 markers is performed by amplification, preferably by PCR, using specific primers which can be used to amplify the resistant/susceptible allele of each of the Cm_MU45437_855, LG5-M2, CMTAN139, and/or LG5-M3 markers.

In particular, detection of the Cm_MU45437_855 marker on LG5 is performed using two forward primers, one being specific for the resistant allele and one being specific for the susceptible allele, and one common reverse primer. Said two forward primers may be selected so as to enable amplifying a nucleic acid comprising or consisting of SEQ ID NO: 34, or a fragment thereof including the [G/A] polymorphism at position 51 of SEQ ID NO: 34. For example, the forward primer for detecting the susceptible allele of the marker Cm_MU45437_855, by amplifying a nucleic acid consisting of sequence SEQ ID NO: 34, may consist of the sequence 5′-AAAGTTTCTGTGTATTAAATCTGAACTCG-3′ (SEQ ID NO: 35), the forward primer for detecting the resistant allele of the marker Cm_MU45437_855, by amplifying a nucleic acid consisting of sequence SEQ ID NO: 34, may consist of the sequence 5′-AATTAAAGTTTCTGTGTATTAAATCTGAACTCA-3′ (SEQ ID NO: 36), and the common reverse primer may consist of the sequence 5′-CAGAGCACGTTTCGAAGGCACATAT-3′ (SEQ ID NO: 37). Using primers consisting of sequences SEQ ID NO: 35, SEQ ID NO: 36 and SEQ ID NO: 37, detection of an adenine (A) rather than a guanine (G) at position 51 of the amplification product consisting of sequence SEQ ID NO: 34 indicates the presence of the QTL on LG5 conferring resistance to PM (see Table 1 below).

In particular, detection of the LG5-M2 marker on LG5 is performed by PCR using a forward primer and a reverse primer which can be used to amplify the resistant/susceptible allele of the LG5-M2 marker. In some embodiments, said PCR is followed by digestion of the amplification products with restriction enzyme or by sequencing the amplification product. In particular, the forward primer and the reverse primer for amplifying the LG5-M2 marker may respectively comprise the sequences 5′-CACTTTCTAAATAGTTTGGAAAAGAG-3′ (SEQ ID NO: 38) and 5′-GAGAATGTCTCTTTATCTAC-3′ (SEQ ID NO: 39). Using primers consisting of SEQ ID NO: 38 and SEQ ID NO: 39, detection of an amplification product of 178 bp indicates presence of the QTL on LG5 conferring resistance to PM (see Table 2 below).

In particular, detection of the CMTAN139 marker on LG5 is performed by PCR using a forward primer and a reverse primer which can be used to amplify the resistant/susceptible allele of the CMTAN139 marker. In some embodiments, said PCR is followed by digestion of the amplification products with restriction enzyme or by sequencing the amplification product. In particular, the forward primer and the reverse primer for amplifying the CMTAN139 marker may respectively comprise the sequences 5′-CGTAGAAGACACACATAATG-3′ (SEQ ID NO: 40) and 5′-GAACTAGAACCACAAATCAC-3′ (SEQ ID NO: 41). Using primers consisting of SEQ ID NO: 40 and SEQ ID NO: 41, detection of an amplification product of 134 bp indicates presence of the QTL on LG5 conferring resistance to PM (see Table 2 below).

In particular, detection of the LG5-M3 marker on LG5 is performed using two forward primers, one being specific for the resistant allele and one being specific for the susceptible allele, and one common reverse primer. Said two forward primers may be selected so as to enable amplifying a nucleic acid comprising or consisting of SEQ ID NO: 42, or a fragment thereof including the [C/T] polymorphism at position 51 of SEQ ID NO: 42. For example, the forward primer for detecting the susceptible allele of the marker LG5-M3, by amplifying a nucleic acid consisting of sequence SEQ ID NO: 42, or its complementary sequence, may consist of the sequence 5′-CAGTCACAGAATTTGTAGTAGACTTATAG-3′ (SEQ ID NO: 43), the forward primer for detecting the resistant allele of the marker LG5-M3, by amplifying a nucleic acid consisting of sequence SEQ ID NO: 42, or its complementary sequence, may consist of the sequence 5′-CAGTCACAGAATTTGTAGTAGACTTATAA-3′ (SEQ ID NO: 44), and the common reverse primer may consist of the sequence 5′-AGAGTTCTTTCTAACGGGCATTGAGATT-3′ (SEQ ID NO: 45). Using primers consisting of sequences SEQ ID NO: 43, SEQ ID NO: 44 and SEQ ID NO: 45, detection of an adenine (A) rather than a guanine (G), or a thymine (T) rather than a cytosine (C) in the complementary strand, at position 51 of the amplification product consisting of sequence SEQ ID NO: 42 indicates the presence of the QTL on LG5 conferring resistance to PM (see Table 1 below).

In some embodiments, said Vat gene analog associated to resistance to aphids on LG5 is identified by amplification product of the forward primer Me-VatE-F having the sequence 5′-CTCCACTCAGAATTGGTAGGTGCC-3′ (SEQ ID NO: 48) and the reverse primer Me-VatE-R having the sequence 5′-CCTTAGAAGAAGATGAAGTCTCCC-3′ (SEQ ID NO: 49). Using this pair of primers, detection of a fragment of 1723 bp indicates the presence of the Vat gene analog.

The alleles conferring the resistances to Scab disease and Powdery Mildew (PM) amplified by the markers defined here above are as described in Table 1 and Table 2.

TABLE 1

| | | | | Position | | Alternative | SEQ |
|---|---|---|---|---|---|---|---|
| | | | Sequence surrounding | in | | alleles | ID |
| Trait | Marker | LG | the marker | SEQ ID | Type | (R/S) | NO |
| Scab disease | Cm_MU45136_209 | 2 | TGCTTGCTGCCGGCCTCCCTGGCGGCCG | 209 | SNP | C/T | 1 |
| | | | CGTGAATTTTATTTAACGGCCCAATCGGC | | | | |
| | | | CCACACATTGCATCAAAAAAAAATATTTCC | | | | |
| | | | CAAGTGTTTCTTAGCATATATTTGTTGTGT | | | | |
| | | | TCAAAGAAACCAAAATATCAAAAGAGGTG | | | | |
| | | | CGCTATTATTTTTTTATCATCGGTTCTTGT | | | | |
| | | | CTCAAGAAGGAAAAAAAAATGTCACTCTT | | | | |
| | | | TCT[T/C]AAGTCTTGCAGCTCCAAGAAATT | | | | |
| | | | TGTTGGTGATCATTCATTA | | | | |
| Scab disease | LG2-M4 | 2 | CATGGAGTGCCGCATACGGTTGATACTTT | 61 | SNP | A/G | 5 |
| | | | GGTTTTCACACCATTTGTAAGTTTGAACTT | | | | |
| | | | T[A/G]CAAACACTAAAGAACTCGTTATCAT | | | | |
| | | | ACGTGCTTGCATTACCTTTCTTGTGATAA | | | | |
| | | | ATGTGT | | | | |
| Scab disease | Cm_MU45398_32 | 2 | AGAATAACAAAACAGGGTTGTTCCGCTTT | 32 | SNP | C/T | 9 |
| | | | AC[T/C]TCAAATAGCAACAAAGAAGAAGAA | | | | |
| | | | GAAGAAGACGATTAGAAGAGAAAGATGG | | | | |
| | | | AAGCGAAGGTCAGCAAATTCTTAGGCTCC | | | | |
| | | | GTTTCCAATTTCTTCTCCGGCGGCGATCA | | | | |
| | | | CATACCATGGTGCGATCGCGACGTCGTT | | | | |
| | | | GCCGGTTGCGAAAGAGAGGCTGCTGAGG | | | | |
| | | | CCGAAAAGAGCTCATCTGGTGAGCTTTTG | | | | |
| | | | AAGGAAAGCATTATGCGTTTATCT | | | | |
| Scab disease | LG5-M1 | 5 | TCCATCCATCTTTTCCAGTTTTGTGGAGTT | 36 | SNP | C/T | 13 |
| | | | CATTA[T/C]GATACAATTTCGCCGATCTAC | | | | |
| | | | TTTTCTTTACAT | | | | |
| Scab disease | Cm_MU46579_322 | 5 | GAAGACTCCGGTAGCTAAAGAAGGCGGT | 51 | SNP | C/T | 16 |
| | | | TCCGATCCTCACTGGAACTATC[T/C]CATG | | | | |
| | | | AAGTTCACAGTCGATGAGGCTGCCTTGC | | | | |
| | | | AGAACAATCGACTCAATC | | | | |
| Scab disease | Cm_MU44050_58 | 5 | GAACCCAAAAAGCTTGGGGGTTTCGTAG | 58 | SNP | C/T | 20 |
| | | | CTTCGTAGCGTTGCTTTCATGGCGAGCTT | | | | |
| | | | [T/C]GTTTCTTTGAGCTTCTCCGTTCCACA | | | | |
| | | | GCTCACTCTCAAGGAAATCCCACCATGGA | | | | |
| | | | AATTGGCTAAAGCTACGGTAGTTTCAAGG | | | | |
| | | | CGAGCTGGAACAGCTTTGAAGCTTGTGC | | | | |
| | | | TACCCTGTTATTCAAAGAAATCCATATCTA | | | | |
| | | | TGAATTGGGAAAACTTTCGATCTGTACGG | | | | |
| | | | TTAACTTCAAGACCTTTTTGGG | | | | |
| PM | LG2-M1 | 2 | TTAATGGAATGACACAGATCAATGTTCTC | 69 | SNP | T/C | 26 |
| | | | ATCTGTTGCCCTCATGGCTTCTGATACTC | | | | |
| | | | GTTCTGATAT[T/C]CCGGGGCCCAAATGA | | | | |
| | | | GGATTGTATGTTCTAACCCATTCTTCGTG | | | | |
| | | | ATTGATTTCGAATTC | | | | |
| PM | Cm_MU47536_461 | 2 | GGTAACATGTGGGAGATTGATGTACAAGA | 51 | SNP | T/A | 30 |
| | | | TTTTGATAATGTGATTGATAC[A/T]AACAT | | | | |
| | | | CAAAGGCTCAGCTAATATTCTTCGTCACT | | | | |
| | | | TCATCCCTCTTATGAT | | | | |
| PM | Cm_MU45437_855 | 5 | CTGAATCATACCAATTGTAATTAAAGTTTC | 51 | SNP | A/G | 34 |
| | | | TGTGTATTAAATCTGAACTC[A/G]AGGCTT | | | | |
| | | | TTCCTCTATATGTGCCTTCGAAACGTGCT | | | | |
| | | | CTGTAACTTGATTTT | | | | |
| PM | LG5-M3 | 5 | ATTTCAAGAGAGTTCTTTCTAACGGGCAT | 51 | SNP | T/C | 42 |
| | | | TGAGATTCCMGAATGGTTCAG[T/C]TATAA | | | | |
| | | | GTCTACTACAAATTCTGTGACTGCTAGCA | | | | |
| | | | TTCGTCACTATCCAGA | | | | |

Markers linked to Scab disease and PM resistance on linkage groups 2 and 5, location and flanking sequences. LG.: Linkage Group, R: Resistant, S: Susceptible, PM: Powdery Mildew.

TABLE 2
resistance allele amplified by the markers LG5-
M3, CMTAN139, CMTCN2, CMBR120 and Me_VatE.

| | | | | | Resistance |
|---|---|---|---|---|---|
| Trait | Marker | Chromosome | Type | Design | allele (bp) |
| Powdery Mildew | LG5-M2 | 5 | SSR | direct labelling | 178 |
| Powdery Mildew | CMTAN139 | 5 | SSR | direct labelling | 134 |
| Scab disease | CMCTN2 | 5 | SSR | indirect labelling | 195 |
| Powdery Mildew | CMBR120 | 2 | SSR | direct labelling | 165 |
| Aphis Gossypii | Me_VatE | 5 | STS | — | 1723 |
| (VatE gene) | | | | | |

In some embodiments, said QTL(s) conferring resistance to Scab disease, PM and Aphids are identified by detection of:
(1) allele C of Cm_MU45136_209, allele A of LG2-M4, and allele C of Cm_MU45398_32,
(2) allele C of LG5-M1, allele of 195 bp of CMCTN2, allele C of Cm_MU46579_322, and allele C of Cm_MU44050_58,
(3) allele of 165 bp of CMBR120, allele T of LG2-M1, and allele T of Cm_MU47536_461,
(4) allele A of Cm_MU45437_855, allele of 178 bp of LG5-M2, allele of 134 bp of CMTAN139, and allele T of LG5-M3, or
(5) allele of 1723 bp of Me_VatE.

Insofar as the QTLs conferring resistance to Scab disease, PM and Aphids can be identified by the specific alleles described in Tables 1 and 2, a plant of the invention preferably comprises any combination of the alleles as defined here above, has a commercially acceptable fruit quality and optionally does not have any necrotic phenotype. For example, the 20 C. melo plant according to the invention may comprises the following combinations of the alleles associated to resistance to Scab disease, PM and Aphids:
I) The allele's combination (1), (3) and (5),
II) The allele's combination (2), (4) and (5),
III) The allele's combination (1), (4) and (5),
IV) The allele's combination (2), (3) and (5),
V) The allele's combination (1), (2), (3) and (5),
VI) The allele's combination (1), (2), (4) and (5),
VII) The allele's combination (1), (3), (4) and (5),
VIII) The allele's combination (2), (3), (4) and (5), or
IX) The allele's combination (1), (2), (3), (4) and (5).

Thus in a particular preferred embodiment, the C. melo plant according to the invention:
comprises the allele's combination IX) as defined here above,
has a commercially acceptable fruit quality, and
optionally does not have any necrotic phenotype linked to the Zym gene.

In some embodiments, said one or more QTLs associated to resistance to Cladosporium, aphids and powdery mildew are chosen from those present in the genome of a plant of the line MTYVVC721, which seeds are deposited under the NCIMB accession number 43317.

In some embodiments, said one or more QTLs associated to resistance to Cladosporium, aphids and powdery mildew are as found in the genome of a plant corresponding to the deposited material MTYVVC721 (NCIMB accession number 43317).

In some embodiments, the C. melo plant according to the invention is line MTYVVC721, which seeds are deposited under NCIMB accession number 43317.

In some embodiments, a plant according to the invention may be a progeny or offspring of a plant grown from the deposited seeds of C. melo line MTYVVC721, deposited at the NCIMB under the accession number 43317. Plants grown from the deposited seeds are indeed homozygously resistant to Scab disease, aphids and powdery mildew, they have a commercially acceptable fruit quality, and does not have any necrotic phenotype linked to the Zym gene, i.e. they thus bear in their genome the QTLs on LG2 and LG5 associated to resistance to Scab disease, aphids and powdery mildew as defined here above at homozygous state; without bearing any necrotic phenotype linked to the Zym gene. They can be used to transfer said QTLs on LG2 and LG5 in another background by crossing and selfing and/or backcrossing, without transferring any necrotic phenotype linked to the Zym gene. A progeny of a plant obtained from the deposited seed can be identified by one skilled in the art, for example by using the markers Cm_MU45136_209, LG2-M4, Cm_MU45398_32, LG5-M1, CMCTN2, Cm_MU46579_322, Cm_MU44050_58, CMBR120, LG2-M1, Cm_MU47536_461, Cm_MU45437_855, LG5-M2, CMTAN139, LG5-M3 and/or Me-VatE. Preferably, such a progeny is identified by at least 2, and more preferably at least 3 of said makers; according to a preferred embodiment, at least one of the markers is associated with the QTL on LG2 or LG5 associated to resistance to Scab disease, at least one of the markers is associated with the QTL on LG2 or LG5 associated to resistance to powdery mildew; a third marker may be a marker associated to resistance to aphids, such as Me-VatE.

The resistance to Scab disease, aphids and powdery mildew is advantageously determined by comparison to a susceptible (commercial) line, for example the Védrantais line. Resistance to Cladosporium is preferably determined as detailed in example 1.1, on the basis of an inoculation test of the plant, at one-leaf stage. Resistance to PM is preferably determined as detailed in example 1.2, either on the basis of an inoculation test of the plant, at a one-leaf stage, or on the basis of a spraying test, applied on detached leaves. Resistance to Aphids is preferably determined as disclosed in the prior art.

According to a second aspect, the present invention is directed to parts of a plant according to the invention.

In some embodiments, a part of a plant is a plant cell. The invention thus provides a cell of a C. melo plant according to the invention, i.e. a plant cell that:
comprises:
   (i) one or more QTL associated to resistance to Scab disease, wherein said one or more QTL maps to linkage group 2 (LG2) and/or linkage group 5 (LG5),
   (ii) one or more QTL associated to resistance to Powdery Mildew, wherein said one or more QTL maps to LG2 and/or LG5, and is different from said one or more QTL in (i), and
   (iii) the Vat gene analog associated to aphids resistance on LG5.

The different features of said QTLs on LG2 and LG5 have been defined in relation with the first aspect of the invention and apply mutatis mutandis to this aspect of the invention. Said QTLs are thus preferably chosen from those present in the genome of a plant corresponding to the deposited material MTYVVC721 (NCIMB accession number 43317). In some embodiments, said QTLs on LG2 and LG5 conferring resistance to Scab disease, aphids and powdery mildew are as found in the genome of a plant corresponding to the deposited material MTYVVC721 (NCIMB accession number 43317).

In some embodiments, said QTLs on LG2 and LG5 conferring resistance to Scab disease, aphids and powdery mildew are as defined in the first aspect of the invention.

In some embodiments, the alleles conferring resistance to Scab disease, aphids and powdery mildew are as described in Tables 1 and 2.

In some embodiments, the plant part according to the invention comprises the allele's combination I) to IX) as defined in the first aspect of the invention.

In some embodiments, the combination of alleles as described here above is as found in the genome of a plant corresponding to the deposited material MTYVVC721 (NCIMB accession number 43317).

A plant cell of the invention may have the capacity to be regenerated into a whole plant, said plant having a commercially acceptable fruit quality, and do not having any necrotic phenotype.

Alternatively, the invention is also directed to plant cells which are not regenerable, and thus are not capable of giving rise to a whole plant.

Preferably, a plant cell according to the invention comprises a QTL conferring resistance to Scab disease that is located on LG2 within a chromosomal region that is delimited by marker Cm_MU45136_209 and marker Cm_MU45398_32, a QTL conferring resistance to Scab disease that is located on LG5 within a chromosomal region delimited by marker LG5-M1 and marker Cm_MU44050_58, a QTL conferring resistance to PM that is located on LG2 within a chromosomal region that is delimited by marker CMBR120 and marker Cm_MU47536_461, a QTL conferring resistance to PM that is located on LG5 within a chromosomal region that is delimited by marker Cm_MU45437_855 and marker LG5-M3, and the Vat gene analog associated to aphids resistance on LG5.

According to another embodiment, the plant part is any other part of a plant according to the invention, it may be in particular seeds, reproductive material, roots, flowers, fruits, rootstock or scion.

All the embodiments detailed in the preceding section in connection with the first aspect of the invention are also preferred embodiments according to this second aspect of the invention.

The invention is more particularly concerned with seed of a C. melo plant, giving rise when grown up to C. melo plant resistant to Scab disease, aphids and powdery mildew as defined above. Such seed are thus ‘seed of a plant of the invention’, i.e. seed giving rise to a plant of the invention. The invention is also concerned with seed from a plant of the invention, i.e. obtained from such a plant after selfing or crossing, provided however that the plant obtained from said seed is resistant to Scab disease, aphids and powdery mildew due to the QTLs on LG2 and LG5 as defined here above conferring said resistance, preferably without bearing any necrotic phenotype linked to the Zym gene.

The present invention is also directed to a tissue culture of regenerable cells of the plant as defined above according to the present invention; preferably, the regenerable cells are derived from embryos, protoplasts, meristematic cells, callus, pollen, leaves, anthers, stems, petioles, roots, root tips, fruits, seeds, flowers, cotyledons, and/or hypocotyls of the invention, and thus comprises in their genome the QTLs on LG2 and LG5 conferring resistance to Scab disease, aphids and powdery mildew as described here above.

The tissue culture will preferably be capable of regenerating plants having the physiological and morphological characteristics of the foregoing C. melo plant, and of regenerating plants having substantially the same genotype as the foregoing C. melo plant. The present invention also provides C. melo plants regenerated from the tissue cultures of the invention.

The invention also provides a protoplast of the plant defined above, or from the tissue culture defined above, said protoplast comprising in its genome the QTLs on LG2 and LG5 conferring resistance to Scab disease, aphids and powdery mildew as described here above.

All the embodiments detailed in the preceding section in connection with the first aspect of the invention are also embodiments according to this second aspect of the invention.

According to a third aspect, the present invention is also directed to the use of a C. melo plant as detailed according to the first aspect of the invention, i.e. resistant to Scab disease, aphids and powdery mildew, as a breeding partner in a breeding program for obtaining C. melo plants resistant to Scab disease, aphids and powdery mildew. Indeed, such a C. melo plant according to the first aspect harbors in its genome QTLs as defined here above conferring said resistances, preferably without bearing any necrotic phenotype linked to the Zym gene. By crossing this plant with susceptible or less resistant plants, it is thus possible to transfer these QTLs, conferring the desired phenotype, to the progeny. A plant according to the invention can thus be used as a breeding partner for introgressing QTLs conferring the desired phenotype into a C. melo plant or germplasm (i.e. without introgressing any necrotic phenotype linked to the Zym gene). The invention is also directed to the same use with plants or seeds of MTYVVC721 as deposited at NCIMB under accession number 43317. Said plants are also suitable as introgression partners in a breeding program aiming at conferring the desired phenotype to a C. melo plant or germplasm.

In such a breeding program, the selection of the progeny displaying the desired phenotype, or bearing sequences linked to the desired phenotype, can advantageously be carried out on the basis of the allele of the markers disclosed here above. The progeny is preferably selected on the presence of one or more of the following specific alleles: allele C of Cm_MU45136_209, allele A of LG2-M4, allele C of Cm_MU45398_32, allele C of LG5-M1, allele of 195 bp of CMCTN2, allele C of Cm_MU46579_322, allele C of Cm_MU44050_58, allele of 165 bp of CMBR120, allele T of LG2-M1, allele T of Cm_MU47536_461, allele A of Cm_MU45437_855, allele of 178 bp of LG5-M2, allele of 134 bp of CMTAN139, allele T of LG5-M3, and allele of 1723 bp of Me_VatE. The progeny is preferably selected on the presence of the allele's combinations I) to IX) as defined in the first aspect of the invention.

The selection of the progeny having the desired phenotype can also be made on conditions of pathogens infestation, as disclosed inter alia in the section Scab test and/or PM test of the examples, or with other tests well-known to the skilled reader.

A plant according to the invention, or as deposited under accession number NCIMB 43317, is thus particularly valuable in a marker assisted selection program for obtaining commercial C. melo lines and varieties resistant to Scab disease, aphids and powdery mildew, but preferably not bearing any necrotic phenotype linked to the Zym gene.

Any embodiment described for the 1^{st} and 2^{nd} aspects of the invention is also applicable to this aspect of the invention.

The invention is also directed to the use of said plants in a program aiming at identifying, sequencing and/or cloning the genetic sequences conferring the desired phenotype.

Any specific embodiment described for the previous aspect of the invention is also applicable to this aspect of the invention, especially with regard to the features of the QTLs conferring the phenotype of interest.

According to another aspect, the invention also concerns methods for the production of C. melo plants resistant to Scab disease, aphids and powdery mildew, especially commercial plants. A method or process for the production of a plant having these features comprises the following steps:
a) Crossing a plant according to the first aspect of the invention (e.g. a plant corresponding to the deposited seeds (NCIMB 43317)) and a susceptible or less resistant C. melo plant, in which the desired phenotype is to be imported or improved,
b) Selecting one plant resistant to Scab disease, aphids and powdery mildew, but preferably not bearing any necrotic phenotype linked to the Zym gene in the progeny thus obtained, or one plant bearing QTLs associated to resistance to Scab disease, aphids and powdery mildew but preferably not bearing any necrotic phenotype linked to the Zym gene,
c) Optionally self-pollinating one or several times the resistant plant obtained at step b) and selecting a plant resistant to Scab disease, aphids and powdery mildew but preferably not bearing any necrotic phenotype linked to the Zym gene in the progeny thus obtained,
d) Backcrossing the resistant plant selected in step b) or c) with a susceptible C. melo plant (i.e. susceptible to Scab disease, aphids and/or powdery mildew), and
e) Selecting a plant resistant to Scab disease, aphids and powdery mildew but preferably not bearing any necrotic phenotype linked to the Zym gene.
   Alternatively, the method or process may comprise the following steps:
a1) Crossing a plant according to the first aspect of the invention (e.g. a plant corresponding to the deposited seeds (NCIMB 43317)) and a susceptible or less resistant C. melo plant, in which the desired phenotype is to be imported or improved, thus generating the F1 population,
a2) selfing the F1 population to create F2 population,
b) Selecting resistant individuals and preferably not bearing any necrotic phenotype linked to the Zym gene in the progeny thus obtained,
c) Optionally self-pollinating one or several times the resistant plant obtained at step b) and selecting a resistant plant preferably not bearing any necrotic phenotype linked to the Zym gene in the progeny thus obtained,
d) Backcrossing the resistant plant selected in step b) or c) with a susceptible C. melo plant (i.e. susceptible to Scab disease, aphids and/or powdery mildew),
e) Selecting a plant resistant to Scab disease, aphids and powdery mildew preferably not bearing any necrotic phenotype linked to the Zym gene.

In some embodiments, plant resistant to Scab disease, aphids and powdery mildew but not bearing any necrotic phenotype linked to the Zym gene can be selected at steps b), c) and e).

The plant selected at step e) is preferably a commercial plant, especially a plant having fruits of type Charentais, Western Shipper, Harper or Italian Cantaloup with a flesh color going from the orange to the red Magenta (i.e. a colorimetry having L*c*h values: 60<L<65, 40<c<50 and 66<h75 as measured by a Minolta colorimeter), a flesh firmness with a mean value of 4 kg/0.5 cm²+/−2 as measured by a penefel, a level of Brix of at least 11°, a diameter of the fruit/size of the cavity ratio as found as in the Vedrantais variety, and a fruit preservation greater than 8 days at +12° C.

Preferably, steps d) and e) are repeated at least twice and preferably three times, not necessarily with the same susceptible C. melo plant. Said susceptible C. melo plant is preferably a breeding line.

The self-pollination and backcrossing steps may be carried out in any order and can be intercalated, for example a backcross can be carried out before and after one or several self-pollinations, and self-pollinations can be envisaged before and after one or several backcrosses.

In some embodiments, such a method is advantageously carried out by using markers as described here above for one or more of the selections carried out at steps b), c) and/or e) for selecting plants resistant to Scab disease, aphids and powdery mildew and preferably not bearing any necrotic phenotype linked to the Zym gene.

In some embodiments, the markers for selecting plants resistant to Scab disease, aphids and powdery mildew and preferably not bearing any necrotic phenotype linked to the Zym gene are:
One or more of the markers Cm_MU45136_209, LG2-M4, Cm_MU45398_32, LG2-M1, Cm_MU47536_461, CMBR120 and Me-VatE, or all the markers Cm_MU45136_209, LG2-M4, Cm_MU45398_32, LG2-M1, Cm_MU47536_461, CMBR120 and Me-VatE,
One or more of the markers LG5-M1, Cm_MU46579_322, Cm_MU44050_58, CMCTN2, Cm_MU45437_855, LG5-M3, LG5-M2, CMTAN139 and Me-VatE, or all the markers LG5-M1, Cm_MU46579_322, Cm_MU44050_58, CMCTN2, Cm_MU45437_855, LG5-M3, LG5-M2, CMTAN139 and Me-VatE,
One or more of the markers Cm_MU45136_209, LG2-M4, Cm_MU45398_32, LG2-M1, Cm_MU47536_461, CMBR120, LG5-M1, Cm_MU46579_322, Cm_MU44050_58, CMCTN2, Cm_MU45437_855, LG5-M3, LG5-M2, CMTAN139 and Me-VatE, or all the markers Cm_MU45136_209, LG2-M4, Cm_MU45398_32, LG2-M1, Cm_MU47536_461, CMBR120, LG5-M1, Cm_MU46579_322, Cm_MU44050_58, CMCTN2, Cm_MU45437_855, LG5-M3, LG5-M2, CMTAN139 and Me-VatE.

In some embodiments, the plant selected at any one of steps b), c) and/or e) is preferably selected on the presence of one of the allele's combination I) to IX) as defined in the first aspect of the invention.

The selection of the progeny having the desired phenotype can also be made on conditions of pathogen infestation, as disclosed inter alia in the section Scab test and/or PM test of the examples or with other tests well-known to the skilled reader.

The method used for allele detection can be based on any technique allowing the distinction between two different alleles of a marker, on a specific chromosome.

The present invention also concerns a C. melo plant obtained or obtainable by such a method. Such a plant is indeed a C. melo plant that is resistant to Scab disease, aphids and powdery mildew according to the first aspect of the invention.

According to a further aspect, the present invention is also directed to hybrid C. melo plants obtainable by crossing a resistant plant according to the first aspect of the invention, such as a plant MTYVVC721, a representative sample of seeds which have been deposited under the NCIMB accession number 43317, or a resistant plant obtainable by the methods disclosed above, with a C. melo plant of, for example a plant susceptible to Scab disease, aphids and powdery mildew infection, or a plant with a different level of resistance to Scab disease, aphids and powdery mildew infection. A particularly preferred hybrid C. melo plant, is a plant which displays any trait or phenotype of agronomical interest.

The invention is also directed to a method for obtaining commercial C. melo plants that are resistant to Scab disease, aphids and powdery mildew, said method comprising the steps of:
Backcrossing a plant obtained by germinating the deposited seeds MTYVVC721 (NCIMB accession number 43317) or a C. melo plant according to the first aspect of the invention, with a C. melo plant, for example a C. melo plant susceptible to Scab disease, aphids and powdery mildew,
Selecting a plant resistant to Scab disease, aphids and powdery mildew that preferably does not bear any necrotic phenotype linked to the Zym gene.

The selection in the second step is preferably carried out as detailed above for the other methods of the invention. Said selection is preferably carried out on the presence of one or more of the specific alleles of the markers and the vat gene analog as described here above, as found in line MTYVVC721.

The plant selected is preferably a commercial plant, especially a plant having fruits of type Charentais, Western Shipper, Harper or Italian Cantaloup with a flesh color going from the orange to the red Magenta (i.e. a colorimetry having L*c*h values: 60<L<65, 40<c<50 and 66<h75 as measured by a Minolta colorimeter), a flesh firmness with a mean value of 4 kg/0.5 cm²+/−2 as measured by a penefel, a level of Brix of at least 11°, a diameter of the fruit/size of the cavity ratio as found as in the Vedrantais variety, and a fruit preservation greater than 8 days at +12° C.

Also provided are methods for producing C. melo plants seeds. In some embodiments, the methods comprise crossing the C. melo plant according to the invention with itself or with another C. melo plant, and harvesting the resultant seeds.

In addition to introgression of the QTLs associated to resistance to Scab disease, aphids and powdery mildew, as detailed in the methods of the invention, said sequences can also be introduced into C. melo background by genetic engineering in order to obtain a commercial C. melo plant resistant to Scab disease, aphids and powdery mildew. The identification and cloning of the introgressed QTLs from C. melo conferring the desired phenotype, inter alia from the deposit, are routine for the skilled person.

According to a further aspect, the present invention provides a plant obtained or obtainable by one of the methods described above. Such a plant is indeed a C. melo plant having the desired phenotype according to the first aspect of the invention, i.e. resistant to Scab disease, aphids and powdery mildew and preferably not having any necrotic phenotype linked to the Zym gene.

It is noted that the seeds or plants of the invention may be obtained by different processes, in particular technical processes such as UV mutagenesis or genetic engineering such as guided recombination, and are not exclusively obtained by means of an essentially biological process.

According to such an aspect, the invention relates to a C. melo plant or seed, preferably a non-naturally occurring C. melo plant or seed, which may comprise one or more mutations in its genome, which provides the mutant plant a resistance to Scab disease, aphids and powdery mildew, which mutation is as present, for example, in the genome of plants of which a representative sample was deposited with the NCIMB under deposit number NCIMB 43317.

Preferably, the mutations are the integration of (i) at least one QTL conferring resistance to Scab disease, wherein said at least one QTL is present on linkage group (LG) 2 and/or linkage group 5 (LG5), (ii) at least one QTL conferring resistance to PM, wherein said at least one QTL is present on LG2 and/or LG5, and is different from said at least one QTL in (i), and (iii) the vat gene analog conferring resistance to aphids on LG5, in replacement of the homologous sequences of a C. melo plant. Even more preferably, the mutation is the (i) substitution of the sequence delimited by marker Cm_MU45136_209 and marker Cm_MU45398_32 on LG2 of a C. melo genome, or a fragment thereof, by the homologous sequence on LG2 present in the genome of a plant of which a representative sample was deposited with the NCIMB under deposit number NCIMB 43317, (ii) substitution of the sequence delimited by marker LG5-M1 and marker Cm_MU44050_58 on LG5 of a C. melo genome, or a fragment thereof, by the homologous sequence on LG5 present in the genome of a plant of which a representative sample was deposited with the NCIMB under deposit number NCIMB 43317, (iii) substitution of the sequence delimited by marker CMBR120 and marker Cm_MU47536_461 on LG2 of a C. melo genome, or a fragment thereof, by the homologous sequence on LG2 present in the genome of a plant of which a representative sample was deposited with the NCIMB under deposit number NCIMB 43317, (iv) substitution of the sequence delimited by marker Cm_MU45437_855 and marker LG5-M3 on LG5 of a C. melo genome, or a fragment thereof, by the homologous sequence on LG5 present in the genome of a plant of which a representative sample was deposited with the NCIMB under deposit number NCIMB 43317, and (v) the vat gene analog, wherein the sequences or fragments thereof confer when combined resistance to Scab disease, aphids and PM.

In an embodiment, the invention relates to a method for obtaining a C. melo plant or seed carrying one or more mutations in its genome, which provides the plant with a resistance to Scab disease aphids and PM. Such a method is illustrated in example 6 and may comprise:
a) treating M0 seeds of a C. melo plant to be modified with a mutagenic agent to obtain M1 seeds;
b) growing plants from the thus obtained M1 seeds to obtain M1 plants;
c) producing M2 seeds by self-fertilisation of M1 plants; and
d) optionally repeating step b) and c) n times to obtain M2+n seeds.

The M2+n seeds are grown into plants and submitted to Scab disease, aphids and PM infection. The surviving plants, or those with the milder symptoms of Scab disease, aphids and PM infection, are multiplied one or more further generations while continuing to be selected for their resistance to Scab disease, aphids and PM. In this method, the M1 seeds of step a) can be obtained via chemical mutagenesis such as EMS mutagenesis. Other chemical mutagenic agents include but are not limited to, diethyl sufate (des), ethyleneimine (ei), propane sultone, N-methyl-N-nitrosourethane (mnu), N-nitroso-N-methylurea (NMU), N-ethyl-N-nitrosourea(enu), and sodium azide. Alternatively, the mutations are induced by means of irradiation, which is for example selected from x-rays, fast neutrons, UV radiation.

In another embodiment of the invention, the mutations are induced by means of genetic engineering. Such mutations also include the integration of sequences conferring the Scab disease, aphids and PM resistance, as well as the substitution of residing sequences by alternative sequences conferring the Scab disease, aphids and PM resistance.

The genetic engineering means which can be used include the use of all such techniques called New Breeding Techniques which are various new technologies developed and/or used to create new characteristics in plants through genetic variation, the aim being targeted mutagenesis, targeted introduction of new genes or gene silencing (RdDM). Example of such new breeding techniques are targeted sequence changes facilitated thru the use of Zinc finger nuclease (ZFN) technology (ZFN-1, ZFN-2 and ZFN-3, see U.S. Pat. No. 9,145,565, incorporated by reference in its entirety), Oligonucleotide directed mutagenesis (ODM), Cisgenesis and intragenesis, RNA-dependent DNA methylation (RdDM, which does not necessarily change nucleotide sequence but can change the biological activity of the sequence), Grafting (on GM rootstock), Reverse breeding, Agro-infiltration (agro-infiltration “sensu stricto”, agro-inoculation, floral dip), 35 Transcription Activator-Like Effector Nucleases (TALENs, see U.S. Pat. Nos. 8,586,363 and 9,181,535, incorporated by reference in their entireties), the CRISPR/Cas system (see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641, which are all hereby incorporated by reference), engineered meganuclease re-engineered homing endonucleases, DNA guided genome editing (Gao et al., Nature Biotechnology (2016), doi: 10.1038/nbt.3547, incorporated by reference in its entirety), and Synthetic 5 genomics). A major part of today's targeted genome editing, another designation for New Breeding Techniques, is the applications to induce a DNA double strand break (DSB) at a selected location in the genome where the modification is intended. Directed repair of the DSB allows for targeted genome editing. Such applications can be utilized to generate mutations (e.g., targeted mutations or precise native gene editing) as well as precise insertion of genes (e.g., cisgenes, intragenes, or transgenes). The applications leading to mutations are often identified as site-directed nuclease (SDN) technology, such as SDN1, SDN2 and SDN3. For SDN1, the outcome is a targeted, non-specific genetic deletion mutation: the position of the DNA DSB is precisely selected, but the DNA repair by the host cell is random and results in small nucleotide deletions, additions or substitutions. For SDN2, a SDN is used to generate a targeted DSB and a DNA repair template (a short DNA sequence identical to the targeted DSB DNA sequence except for one or a few nucleotide changes) is used to repair the DSB: this results in a targeted and predetermined point mutation in the desired gene of interest. As to the SDN3, the SDN is used along with a DNA repair template that contains new DNA sequence (e.g. gene). The outcome of the technology would be the integration of that DNA sequence into the plant genome. The most likely application illustrating the use of SDN3 would be the insertion of cisgenic, intragenic, or transgenic expression cassettes at a selected genome location. A complete description of each of these techniques can be found in the report made by the Joint Research Center (JRC) Institute for Prospective Technological Studies of the European Commission in 2011 and titled “New plant breeding techniques—State-of-the-art and prospects for commercial development”, which is incorporated by reference in its entirety.

The present invention also provides methods for detecting and/or selecting a C. melo plant that is resistant to Scab disease, aphids and powdery mildew (PM), and that optionally does not have any necrotic phenotype linked to the Zym gene, wherein said method comprises the steps of:
(i) detecting the presence of at least one QTL conferring resistance to Scab disease, wherein said at least one QTL is present on linkage group (LG) 2 and/or linkage group 5 (LG5),
(ii) at least one QTL conferring resistance to PM, wherein said at least one QTL is present on LG2 and/or LG5, and is different from said at least one QTL in (i), and (iii) detecting the presence of the Vat gene analog associated to aphids resistance on LG5, and
(iv) optionally detecting the absence of the necrotic phenotype linked to the Zym gene.

Preferably, said QTL conferring resistance to Scab disease that is present on LG2 is located within a chromosomal region that is delimited by marker Cm_MU45136_209 and marker Cm_MU45398_32. In some embodiments, said QTL that is present on LG2 can be identified by amplifying any one of the following markers: Cm_MU45136_209, LG2-M4, and Cm_MU45398_32; or any other markers within the chromosomal region delimited by marker Cm_MU45136_209 and marker Cm_MU45398_32.

Preferably, said QTL conferring resistance to Scab disease that is present on LG5 is located within a chromosomal region that is delimited by marker LG5-M1 and marker Cm_MU44050_58. In some embodiments, said QTL that is present on LG5 can be identified by amplifying any one of the following markers: LG5-M1, CMCTN2, Cm_MU46579_322, and Cm_MU44050_58; or any other markers within the chromosomal region delimited by marker LG5-M1 and marker Cm_MU44050_58.

Preferably, said QTL conferring resistance to PM that is present on LG2 is located within a chromosomal region that is delimited by marker CMBR120 and marker Cm_MU47536_461. In some embodiments, said QTL that is present on LG2 can be identified by amplifying any one of the following markers: CMBR120, LG2-M1, and Cm_MU47536_461; or any other markers within the chromosomal region delimited by marker CMBR120 and marker Cm_MU47536_461.

Preferably, said QTL conferring resistance to PM that is present on LG5 is located within a chromosomal region that is delimited by marker Cm_MU45437_855 and marker LG5-M3. In some embodiments, said QTL that is present on LG5 can be identified by amplifying any one of the following markers: Cm_MU45437_855, LG5-M2, CMTAN139, and LG5-M3; or any other markers within the chromosomal region delimited by marker Cm_MU45437_855 and marker LG5-M3.

According to a preferred embodiment, a plant according to the invention does not comprise homozygously the Zym gene on LG2. A plant of the invention may however comprise heterozygously such a gene providing resistance to potyviruses, especially ZYMV. The methods or processes of the invention therefore preferably also comprise a step of detecting whether the Zym gene is present.

As detailed in the experimental section of the application, especially example 5, presence of the Zym gene, providing resistance, can be detected by the SNP marker LG2-M2 (SEQ ID NO: 50), Cm-MU47380_465 (also called MU47380_465, SEQ ID NO: 54) and/or LG2-M3 (SEQ ID NO: 58). Preferably, said QTL or gene conferring resistance to ZYMV that is present on LG2, is located within a chromosomal region that is delimited by marker LG2-M2 and LG2-M3. In some embodiments, said QTL that is present on LG2 can be identified by amplifying any one of the following markers: LG2-M2, Cm-MU47380_465 and LG2-M3; or any other markers within the chromosomal region delimited by marker LG2-M2 and marker LG2-M3. According to the invention, a plant is selected if it comprises at least one of the following allele: allele C of LG2-M2, allele G of MU47380_465 or allele A of LG2-M3. These alleles represent indeed the absence of the QTL or gene conferring resistance to potyviruses, detection of one or more of these alleles thus indicates that the Zym gene is not homozygously present, thus ensuring the absence of the necrotic phenotype linked to the Zym gene.

The markers and the alleles linked to the resistance to ZYMV are as described in Table D. Potential primers which can be used to amplify the marker sequences and discrimination between the different alleles of the SNPs are reported in table G.

In some embodiments, a plant is selected if any one of the allele's combination I) to IX), as defined in the first aspect of the invention, is detected in a genetic material sample of the plant to be selected. Preferably, a plant is selected if the allele's combination the allele's combination IX), as defined in the first aspect of the invention, is detected in a genetic material sample of the plant to be selected.

Preferably, in addition to the allele's combinations I) to IX) as defined in the first aspect of the invention, a plant is detected if one of the alleles representative of the absence of Zym gene is detected.

In some embodiments, detection of the markers Cm_MU45136_209, LG2-M4, Cm_MU45398_32, LG5-M1, Cm_MU46579_322, Cm_MU44050_58, LG2-M1, Cm_MU47536_461, Cm_MU45437_855 and/or LG5-M3 is performed by amplification, e.g. by PCR, using, for each marker, one forward primer which can be used for amplifying the resistant allele, one forward primer which can be used for amplifying the susceptible allele and one common reverse primer. In particular, the primers for amplifying each of said markers may have the sequences as described in the first aspect of the invention, and detailed in table G. The same applies to the markers linked to the Zym gene disclosed above; potential primers are disclosed in table G.

In a preferred embodiment, the amplification is as described in the examples. In a still preferred embodiment, the amplification is performed using a two-step touchdown method in which the elongation and annealing steps are incorporated into a single step. The temperature used for the annealing stage determines the specificity of the reaction and hence the ability of the primers to anneal to the DNA template. A touchdown PCR involves a first step of Taq polymerase activation, followed by a second step called the touchdown step that involves a high annealing temperature and incrementally decreasing the annealing temperature in each PCR cycle, and a third step of DNA amplification. The higher annealing temperatures in the early cycles of a touchdown ensure that only very specific base pairing will occur between the DNA and the primer, hence the first sequence to be amplified is most likely to be the sequence of interest. The annealing temperature is gradually decreased to increase the efficiency of the reaction. The regions that were originally amplified during the highly specific early touchdown cycles will be further amplified and outcompete any non-specific amplification that may occur at the lower temperatures.

In another embodiment, the amplification of SNP markers is performed as recommended in the KASPar assay and illustrated in the examples (see example 5), namely by PCR cycles, comprising a first denaturation step at 94° C. during around 15 minutes, at least 10 cycles of around 20 seconds at 94° C. followed by around 60 second at a decreasing temperature from 65° C. for the 1^{st} cycle to 57° C. for the last cycle, and around 35 cycles of around 20 seconds at 94° C. followed by around 60 seconds at 57° C. This protocol can easily be adapted by a skilled person, depending on the type of primers used.

In some embodiments, detection of the markers LG5-M2, CMTAN139, CMCTN2, CMBR120, and/or Me_VatE is performed by amplification, e.g. by PCR, using, for each marker, one forward primer and one reverse primer. In particular, the primers for amplifying each of said markers may have the sequences as described in the first aspect of the invention.

The present invention is also directed to hybrid C. melo plant, obtained or obtainable by crossing a C. melo plant according to the first aspect of the invention, or a resistant plant obtained or obtainable by the method disclosed here above, with a C. melo plant, for example a C. melo plant susceptible to Scab disease, Aphids, and PM, or a C. melo plant with a different level of resistance to Scab disease, Aphids, and PM.

According to a further aspect, the present invention also provides molecular markers that are linked to the QTL on LG2 and/or LG5 as defined here above conferring the resistance to Scab disease, aphids and/or Powdery Mildew (PM).

In some embodiments, said molecular markers linked to the QTL conferring the resistance to Scab disease on LG2 are one or more of the markers Cm_MU45136_209, LG2-M4, and Cm_MU45398_32, or all the markers Cm_MU45136_209, LG2-M4, and Cm_MU45398_32, or a combination of the markers Cm_MU45136_209, LG2-M4, and Cm_MU45398_32, or any other markers within the chromosomal region delimited by marker Cm_MU45136_209 and marker Cm_MU45398_32.

In some embodiments, said molecular markers linked to the QTL conferring the resistance to Scab disease on LG5 are one or more of the markers LG5-M1, CMCTN2, Cm_MU46579_322, and Cm_MU44050_58, or all the markers LG5-M1, CMCTN2, Cm_MU46579_322, and Cm_MU44050_58, or a combination of the markers LG5-M1, CMCTN2, Cm_MU46579_322, and Cm_MU44050_58, or any other markers within the chromosomal region delimited by marker LG5-M1 and marker Cm_MU44050_58.

In some embodiments, said molecular markers linked to the QTL conferring the resistance to PM on LG2 are one or more of the markers CMBR120, LG2-M1, and Cm_MU47536_461, or all the markers CMBR120, LG2-M1, and Cm_MU47536_461, or a combination of the markers CMBR120, LG2-M1, and Cm_MU47536_461, orany other markers within the chromosomal region delimited by marker CMBR120 and marker Cm_MU47536_461.

In some embodiments, said molecular markers linked to the QTL conferring the resistance to PM on LG5 are one or more of the markers Cm_MU45437_855, LG5-M2, CMTAN139, and LG5-M3, or all the markers Cm_MU45437_855, LG5-M2, CMTAN139, and LG5-M3, or a combination of the markers Cm_MU45437_855, LG5-M2, CMTAN139, and LG5-M3, or any other markers within the chromosomal region delimited by marker Cm_MU45437_855 and marker LG5-M3.

The sequences of the markers as mentioned above are described in Tables 1 and 2, and in the examples.

Further provided is the use of one or more of the molecular markers Cm_MU45136_209, LG2-M4, Cm_MU45398_32, LG5-M1, CMCTN2, Cm_MU46579_322, Cm_MU44050_58, CMBR120, LG2-M1, Cm_MU47536_461, Cm_MU45437_855, LG5-M2, CMTAN139 and LG5-M3 or all the markers Cm_MU45136_209, LG2-M4, Cm_MU45398_32, LG5-M1, CMCTN2, Cm_MU46579_322, Cm_MU44050_58, CMBR120, LG2-M1, Cm_MU47536_461, Cm_MU45437_855, LG5-M2, CMTAN139 and LG5-M3, or a combination of the markers Cm_MU45136_209, LG2-M4, and Cm_MU45398_32, LG5-M1, CMCTN2, Cm_MU46579_322, Cm_MU44050_58, CMBR120, LG2-M1, Cm_MU47536_461, Cm_MU45437_855, LG5-M2, CMTAN139, and LG5-M3, for detecting a C. melo plant that is resistant to Scab disease, aphids and/or Powdery Mildew (PM). Said use may also additionally comprise the use of one or more or all of the SNP markers LG2-M2, Cm-MU47380_and LG2-M3, for detecting a C. melo plant not exhibiting the necrotic phenotype linked to the Zym gene.

The invention is also directed to the use of at least one of the markers of the list Cm_MU45136_209, LG2-M4, Cm_MU45398_32, CMBR120, LG2-M1, Cm_MU47536_461, LG5-M1, CMCTN2, Cm_MU46579_322, Cm_MU44050_58, Cm_MU45437_855, LG5-M2, CMTAN139 and LG5-M3, associated with QTLs on LG2 (the 1st to 6^{th} SNP of the list) and LG5 (the 7^{th} to 14^{th} SN P of the list) conferring the resistance to Scab disease, aphids and/or Powdery Mildew (PM) according to the invention, for identifying alternative molecular markers associated with said QTLs, wherein said alternative molecular markers are:
in the chromosomal region delimited on LG2 by marker Cm_MU45136_209 and marker Cm_MU45398_32, or by marker CMBR120 and marker Cm_MU47536_461,
in the chromosomal region delimited on LG5 by marker LG5-M1 and marker Cm_MU44050_58, or by marker Cm_MU45437_855 and marker LG5-M3,
at less than 2 megabase units from the locus of the 14 markers of the invention, namely Cm_MU45136_209, LG2-M4, Cm_MU45398_32, CMBR120, LG2-M1, Cm_MU47536_461, LG5-M1, CMCTN2, Cm_MU46579_322, Cm_MU44050_58, Cm_MU45437_855, LG5-M2, CMTAN139 and LG5-M3.

The alternative molecular markers are preferably associated with said QTL(s) with a p-value of 0.05 or less, preferably less than 0.01. The QTLs are to be found in the deposited seeds NCIMB 43317.

The invention is also directed to a method for identifying a molecular marker associated with a QTL conferring resistance to Scab disease, aphids and/or Powdery Mildew (PM) when present heterozygously or homozygously, comprising:
identifying a molecular marker in the chromosomal region:
   delimited on LG2 by marker Cm_MU45136_209 and marker Cm_MU45398_32, or by marker CMBR120 and marker Cm_MU47536_461,
   elimited on LG5 by marker LG5-M1 and marker Cm_MU44050_58, or by marker Cm_MU45437_855 and marker LG5-M3, or
   at less than 2 megabase units from the locus of the 15 SNPs markers of the invention, namely Cm_MU45136_209, LG2-M4, Cm_MU45398_32, CMBR120, LG2-M1, Cm_MU47536_461, LG5-M1, CMCTN2, Cm_MU46579_322, Cm_MU44050_58, Cm_MU45437_855, LG5-M2, CMTAN139 and LG5-M3, and
determining whether said molecular marker is associated with or linked to the resistance to Scab disease, aphids and/or Powdery Mildew (PM) in a segregating population issued from a plant exhibiting said resistance.

The population is preferably issued from a plant grown from the deposited seeds NCIMB 43317 or from a progeny thereof, exhibiting the resistance to Scab disease, aphids and/or Powdery Mildew (PM) as described in the invention.

The QTLs on LG2 and LG5 mentioned above, conferring the resistance to Scab disease, aphids and/or Powdery Mildew (PM) according to the invention, are the QTLs present in MTYVVC721 (NCIMB 43317).

Genetic association or linkage is as defined above; preferably the association or linkage is with a p-value of preferably less than 0.05, and most preferably less than 0.01 or even less.

A molecular marker and the resistance phenotype are inherited together in preferably more than 90% of the meiosis, preferably more than 95%.

In a further aspect, the invention relates to method for the production of C. melo plantlets or plants resistant to Scab disease, aphids and Powdery Mildew (PM), which method comprises:
i. culturing in vitro an isolated cell or tissue of the C. melo plant according to the invention to produce C. melo micro-plantlets resistant to Scab disease, aphids and Powdery Mildew (PM), and
ii. optionally further subjecting the C. melo micro-plantlets to an in vivo culture phase to develop into C. melo plant resistant to Scab disease, aphids and Powdery Mildew (PM).

The isolated cell or tissue used to produce a micro-plantlet is an explant obtained under sterile conditions from a C. melo parent plant of the invention to be propagated. The explant comprises or consists, for instance, of a cotyledon, hypocotyl, stem tissue, leaf, embryo, meristem, node bud, shoot apice, or protoplast. The explant can be surface sterilized before being placed on a culture medium for micropropagation.

Conditions and culture media that can be suitably used in plant micropropagation are well known to those skilled in the art of plant cultivation and are described, for example, in “Plant Propagation by Tissue Culture, Handbook and Directory of Commercial Laboratories, eds. Edwin F George and Paul D Sherrington, Exegetics Ltd, 1984”.

Micropropagation typically involves:
i. axillary shoot production: axillary shoot proliferation is induced by adding cytokinin to the shoot culture medium, to produce shoots preferably with minimum callus formation;
ii. adventitious shoot production: addition of auxin to the medium induces root formation, in order to produce plantlets that are able to be transferred into the soil. Alternatively, root formation can be induced directly into the soil.

Plantlets can be further subjected an in vivo culture phase, by culture into the soil under lab conditions, and then progressive adaptation to natural climate, to develop into C. melo plant resistant to Scab disease, aphids and Powdery Mildew (PM).

In view of the ability of the resistant plants of the invention to restrict the damages caused by Scab disease, aphids and PM, they are advantageously grown in an environment infested or likely to be infested or infected by aphids, Cladosporium, Podosphaera xanthii and/or Golovinomyces cichoracearum; in these conditions, the resistant plants of the invention produce more marketable melons than susceptible plants. The invention is thus also directed to a method for improving the yield of C.melo plants or for increasing the number of harvestable C. melo plants or fruits, in an environment infested by Scab disease, aphids and Powdery Mildew (PM) comprising growing in said environment C. melo plants resistant to Scab disease, aphids and PM as defined, comprising on LG2 and/or LG5 the QTLs or sequences according to the invention and conferring to said plants resistance to Scab disease, aphids and Powdery Mildew (PM).

Preferably, the method comprises a first step of choosing or selecting a C. melo plant comprising said sequences of interest conferring to said plants resistance to Scab disease, aphids and PM, preferably without any necrotic phenotype linked to the Zym gene. The method can also be defined as a method of increasing the productivity of a C. melo field, tunnel or glasshouse, or as a method of reducing the intensity or number of chemical or fungicide applications in the production of melons.

The invention is also directed to a method for reducing the loss on C. melo production in condition of Scab disease, aphids and PM infestation or infection, comprising growing a C. melo plant as defined above.

The resistant plants of the invention are also able to restrict the growth of the pathogens responsible for Scab disease and PM, and of aphids, thus limiting the infection of further plants and the propagation of the pathogens and aphids. Accordingly, the invention is also directed to a method for protecting a field, tunnel or glasshouse, or any other type of plantation, from Scab disease, aphids and PM infestation, or of at least limiting the level of infestation or limiting the spread of Scab disease, aphids and PM. Such a method preferably comprises the step of growing a resistant or tolerant plant of the invention, i.e. a plant comprising on LG2 and/or LG5 the sequences conferring resistance to Scab disease, aphids and PM, preferably without any necrotic phenotype linked to the Zym gene.

The invention also concerns the use of a C. melo plant resistant to Scab disease, aphids and PM, according to the invention, for controlling infestation by Scab disease, aphids and PM, in a field, tunnel or glasshouse, or other plantation.

All the preferred features of the QTL are as defined in connection with the other aspects of the invention, namely it is preferably present in the seeds of MTYVVC721 (NCIMB accession number 43317), and it is identifiable by the markers as defined according to the present invention.

The present invention is also directed to a method for improving the yield of C. melo plants in an environment infested by Scab disease, aphids and Powdery Mildew (PM) comprising:
a. identifying C. melo plants resistant to Scab disease, aphids and PM comprising in their genome (i) at least one QTL conferring resistance to Cladosporium, wherein said at least one QTL is present on linkage group (LG) 2 and/or linkage group 5 (LG5), (ii) at least one QTL conferring resistance to PM, wherein said at least one QTL is present on LG2 and/or LG5, and is different from said at least one QTL in (i), and (iii) the vat gene analog conferring resistance to aphids on LG5, and
b. growing said resistant C. melo plants in said infested environment.

By this method, the yield if the C. melo plants is increased, inter alia more marketable melon can be harvested, or more commercial melons are produced, or more seeds are obtained.

In still a further aspect, the invention also relates to a method of producing melons comprising:
a) growing a C. melo plant of the invention, as defined previously;
b) allowing said plant to set fruit; and
c) harvesting fruit of said plant, preferably at maturity and/or before maturity.

All the preferred embodiments regarding the C. melo plant are already disclosed in the context of the previous aspects of the invention.

The method may advantageously comprise a further step of processing said melons into a processed food.

Throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”).

### Seed Deposit

A representative sample of seeds from the C. melo plant according to the invention (i.e. seeds from MTYVVC721 plant) has been deposited by HM-Clause, S. A., Rue Louis Saillant, Z. I. La Motte, BP83, 26802 Portes-lés-Valence cedex, France, pursuant to, and in satisfaction of, the requirements of the Budapest Treaty on the International Recognition of the Deposit of Microorganisms for the Purposes of Patent Procedure (the “Budapest Treaty”) with the National Collection of Industrial, Food and Marine Bacteria (NCIMB), 23 St Machar Drive, Aberdeen, Scotland, AB21 9YA, United Kingdom, on December 13^{th}, 2018, under accession number 43317.

A deposit of the MTYVVC721 seeds is maintained by HM-Clause, S. A., Rue Louis Saillant, Z. I. La Motte, BP83, 26802 Portes-les-Valence cedex, France.

### EXAMPLES

The objective of the following experiments was to obtain, thanks to molecular markers, a new diseases resistance package, useful in breeding lines and commercial hybrids, cumulating two resistance QTLs from two wild accessions on linkage group 5 (one Scab resistance QTL and one PM/vat resistance QTL) and also two resistance QTLs on LG2 (one PM resistance QTL from a wild accession and on scab resistance QTL, identified in a Charentais line), thus four resistance QTLs in total, on 2 linkage groups.

With the help of markers, tests including PM artificial tests, many cycles of selfing and backcrosses (8 breeding cycles), with high number of plants for some cycles (from 24 to more than 100), the inventors succeeded to introgress 2 small chromosomal regions from a wild accession genome and to limit the occurrence of linkage drag related to the shape and the rind color of the fruit.

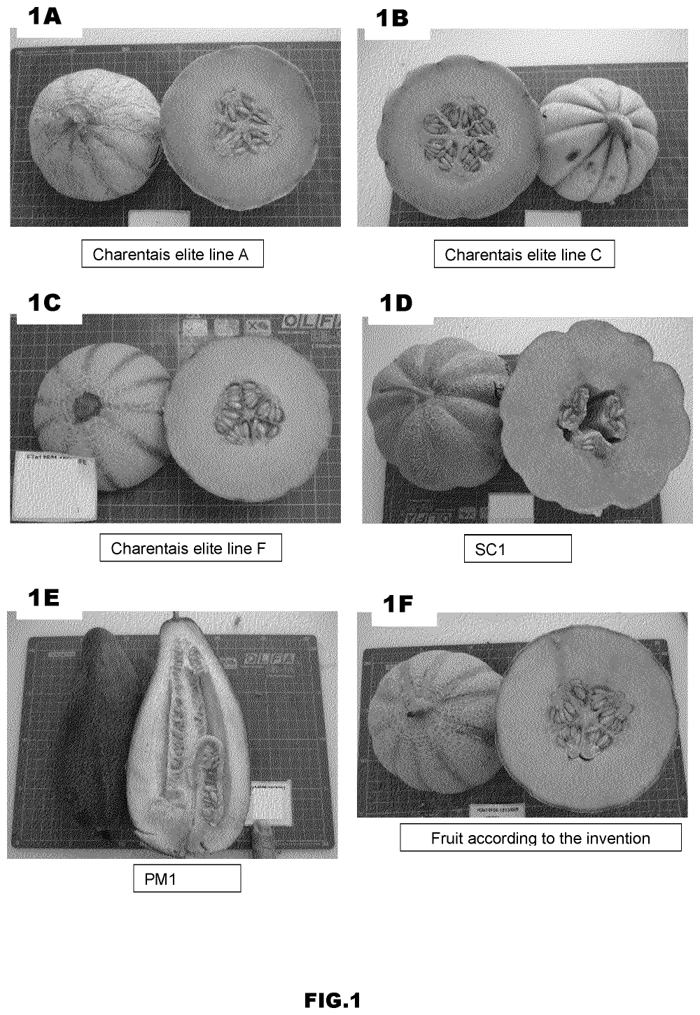

To introduce the SCAB resistance into elite breeding line the inventors have used a wild landrace and had to discard all the undesirable traits linked to this landrace. This melon indeed harbors an undesirable shape (see FIG. 1D) with a pale flesh color and a big cavity. The brix level is also rather low, around 10-11° B at full maturity and the flesh become very soft rapidly after the climacteric crisis. The inventors have nevertheless been able to recover internal qualities, the shelf life potential, the fruit look and shape from Charentais elite line.

Moreover, using an artificial pathology test on the backcrossed plants in addition to the use of flanking markers linked to the 2 identified QTLs on the 2 linkage groups (LG) carriers (LG2 and LG5) and random markers on non-carrying LG, the recovery of the Charentais background was rendered possible.

For each backcross or selfing cycle the inventors have managed between 100 to 300 plants with these different tools plus a fruit assessment to discard the undesirable ones (too elongated shape, soft and pale flesh, grey/green rind that become orange at maturity . . . . ), keeping the plants with the most desirable Charentais elite line traits.

Thanks to the flanking markers and the high number of plants screened with the Cladosporium test and then markers analysis, the inventors have introgressed a small genome fragments from the wild accessions, avoiding the undesirable traits from these melon accessions (cavity size, flesh firmness, flesh color . . . ), as well as the necrosis symptoms disclose in the art as linked to the PM and SCAB resistances.

### 1. Material and Methods

### 1.1. Cladosporium Test (or Scab Test).

The Cladosporium cucumerinum strain used for the test was conserved at −80° C. The inoculum is prepared by culturing the fungus during 14 days before inoculation.

For each tested line or genotype, 20 different seeds/plants are tested, as well as 2 susceptible controls and two resistant controls.

The test is carried out on plants at 1-leaf stage of growth (i.e. around 10 to 14 days after sowing). For the inoculation, conidia are suspended in water and filtered, to arrive at a concentration of 104 to 105 conidia per ml. The inoculation is carried out by spraying the inoculum on the leaf of the plants to be tested. The plants are then cultured on conditions corresponding to 18° C. night/22° C. day with 14 hours of daylight, under satured humidity conditions during the first days.

The results of the scab test are read (1^{st} reading) 7 days after inoculation, and a second reading is made 5 days after the 1^{st} reading with a 1 to 9 scoring scale for symptoms, as follows:

| | Resistance | |
|---|---|---|
| | Score | Symptom description |
| Susceptible (S) | 1 | Death of plant |
| Susceptible (S) | 3 | Necrosis lesions (bigger than spots) |
| | | affecting the foliar part of the plant |
| | | (leaves, cotyledons, petiols) |
| Intermediate (IR) | 5 | Few necrosis spots on apex and beginning |
| | | on cotyledons and petiols |
| Resistant (R) | 7 | Few necrosis spots on apex |
| Resistant (R) | 9 | No symptom |

The Disease Index (DI) of a population of plants is calculated on the basis of the Resistance Score of the individual plants, as follow:

DI=[(0×nb of plants have a resistance score of 1)+(3×Nb of plants having resistance score of 3)+(5×Nb of plants having resistance score of 5)+(7×Nb of plants having resistance score of 7)+(9×Nb of plants having resistance score of 9)]/(9×total number of plants).

If DI=1, all the plants are resistant.
If DI=0, all the plants are susceptible.

### 1.2. Powdery Mildew Test

### Initial Test Leaf Disk Assay:

Different fungi can cause powdery mildew on melons, including Podosphaera xanthii (Px) and Golovinomyces cichoracearum var. cichoracearum (Gc). Insofar as these fungi are obligate fungi, they were maintained on susceptible zucchinis and melon plants, namely Tosca and Edisto varieties respectively.

The test is carried out on plants at 1-leaf stage of growth, when the second leaf is appearing (i.e. around 11 to 13 days after sowing).

For each tested line or genotype, 24 different seeds/plants are tested, as well as 8 different controls, from the more susceptible to the more resistant: Védrantais, Nantais oblong, PMR 45, WMR 29, Edisto 47, PMR 5, PI 124112, MR1 and PM1.

For the inoculation, conidia on zucchinis are suspended in water, to arrive at a concentration of 104 to 105 conidia per ml. The inoculation is carried out by spraying the inoculum on the leaves of the plants to be tested. A second inoculation is carried out 3 days after the first one, also by spraying the inoculum on the leaves.

Results reading: Reading is made 10 days after inoculation. A second reading is made 5 to 6 days after the 1^{st} reading.

The scale of the symptoms is as follow (resistance score):

| | Mycelial growth and sporulation | | |
|---|---|---|---|
| Score | of the fungus on the leaf surface | Other organs | |
| 1 | 50% or more of the leaf | Intense sporulation | Susceptible |
| | surface with mycelium or spores | | |
| 3 | 25 to 50% of the leaf surface | Several spots of | |
| | | sporulation on | |
| | | cotyledons and first leaf | |
| 5 | 5 to 25% of the leaf surface | Few spots of sporulation | Intermediate |
| | | on cotyledons and first | Resistant |
| | | leaf | |
| 7 | Up to 5% | Very few light spots of | Resistant |
| | | sporulation on | |
| | | cotyledons and first leaf | |
| 9 | No symptom | Cotyledons: no | Highly |
| | | symptoms or necrosis | resistant |

Improved Test Detached Leaf Test:

In order to improve the speed of the PM testing, a second test was designed, applied on detached leaves. For this test, 15 different plants of the progeny under study are simultaneously tested, with two replicates per plant (R1 and R2), and with two lectures per plant (Lect 1 and Lect 2). Control plants are tested on 1 or 2 plants, with two replicates per plant (R1 and R2) and with two lectures per plant (Lect 1 and Lect 2). The inoculation of isolated Podosphera xanthii races (Px1, Px2, Px3, Px5 and Px3-5) and Goloivinomyces cichoracearum race 1 (GC) has been made by spraying each race on foliar disc, spread on an agar plate. The occurrence of sporulation in each disc is done twice, Read 1 and Read 2, respectively at 9 days and 11 days after inoculation. The scoring scale (susceptibility scoring scale) is as follow: 0: no sporulation (Resistant), 1: light sporulation <10% of foliar disc (Resistant), 3: sporulation <30% of foliar disc (Intermediate), 5: sporulation <60% of foliar disc (Intermediate), 7: sporulation >60% of foliar disc (Susceptible), 9: full sporulation of the disc (Susceptible).

Of note, the susceptibility score determined according to this testing has been adjusted such that it fits the following relation:

“susceptibility score”=9−“resistance score”.

### 1.3. Necrosis Test:

The occurrence of necrosis was tested in glasshouses, with high humidity. The plants were classified as exhibiting necrosis or not.
By “necrotic phenotype linked to the Zym gene”, it is meant the necrotic phenotype as described in Pitrat and Lecoq, 1984, Euphytica, 33(1):57-61. More specifically, such a necrotic phenotype corresponds to the appearance of necrotic spots in the epidermis of the leaf which extent gradually on the leaf and can lead to completely drying the leaf or of the stem.

### Virus Isolation and Maintenance:

The necrosis phenotype was evaluated by inoculating melon plants with an isolate of Zucchini Yellow Mosaic Potyvirus (ZYMV), stored in plastic bag at −80° C. or on dessicated infected leaves at +4° C. The source of inoculum is propagated on melon plantlets according to mechanical inoculation process.

### Mechanical Inoculation:

The inoculum was made by grinding 1 g of infected leaves with 4 ml of a 0.03M Na₂HPO4 buffer containing 0.2% sodium diethyldithiocarbamate with carborundum (7.5%) and activated carbon (10%). Seedlings with first expanded leaf were inoculated on both cotyledons.
The test is conducted in growth chamber under a photoperiod of 14 h of daylight at 24° C. and 10 h of night at 20° C.

### Symptoms Evaluation:

The evaluation is performed when the susceptible control shows symptoms, about 10 to 14 days after inoculation.
One ‘necrotic’ control (carrying the Fn gene); one ‘mosaic’ control (not carrying the gene) and resistant controls are included in the test to validate the test and check the virus virulence.
Evaluation is done on leaves according and ordinal scale: 1: plant death, complete plant wilting; 3: obvious vein clearing; 5: mild mosaic; 7: light chlorotic spots on young leaves; 9: no symptom at all.
A second scoring can be done few days later to check any symptom evolution.

### 1.4. Melon Accession and Lines

The Charentais elite line C (FIG. 1B) belongs to the Cucumis melo L. subsp. melo species. This elite line is susceptible to P. xanthii races Px-1, Px-2, Px-3, Px-5 and Px3-5 and has a dark orange and firm flesh at maturity, a dark green suture, a yellowing smooth rind, a good shelf life and a high sugar level (15-16° Brix).

The Charentais elite line A (FIG. 1A) belongs to the Cucumis melo L. subsp. melo species. This elite line is susceptible to C. cucumerinum and tolerant to Powdery Mildew and has an orange and firm flesh at maturity, a dark green suture, few yellowing lightly netted rind, a good shelf life and a high sugar level (15-16° Brix).

The Charentais hybrid B belongs to the Cucumis melo L. subsp. melo species. This hybrid harbors high internal quality (nice orange flesh color with high Brix (15-16°) with denser netting, earliness and good shelf life.

The melon accession PM1 (FIG. 1E) belongs to the Cucumis melo sp. Momordica species. This accession is resistant to P. xanthii races Px-1, Px-2, Px-3, Px-5 and Px3-5 and has pale and soft flesh at maturity, big cavity, high yellowing/orange rind at maturity and low level of sugar (10° Brix).

The melon accession SC1 (FIG. 1D) belongs to the Cucumis melo L. subsp. melo species. This landrace is resistant to C. cucumerinum and has a pale orange and soft flesh at maturity, a big cavity, a ribby shape, high yellowing rind and a low level of sugar (10° Brix).

The Charentais elite line F (FIG. 1C) belongs to the Cucumis melo L. subsp. melo species. This elite line is susceptible to SCAB and PM. It is a monoecious line, well rounded, well netted with a nice dark green suture, with medium internal quality, orange flesh, mid firm, average BRIX 12-14° C., classified as early maturing line with a yellowing rind.

### 1.5. DNA Extraction and Genotyping Protocol

### DNA Purification

Genomic DNA automated isolation was performed from 1st young leave tissues using the NucleoMag® 96 plant kit (Macherey Nagel) according to the manufacturer's instructions.
SSR genotyping was carried out according to the methods well known to the skilled reader. Appropriate primers are disclosed in Table E below.
SNP genotyping was also carried out according to well-known method, especially determination of the allele by the KASPar assay (KBioscience Competitive Allele-Specific Polymerase chain reaction assay). This SNP genotyping assays from KBioscience is based on competitive allele specific PCR (one primer per allele) and on FRET (Fluorescent Resonance Energy Transfer) for the detection of SNP without the need for a separation step.
A KASPar mix is prepared, for each SNPs, comprising two competitive, allele-specific forward primers and one common reverse primer. The KASP Assay mix is specific to the SNP to be targeted. The KASP Assay mix is mixed with the DNA sample to be tested and with the KASP Master Mix® containing the universal FRET cassettes, ROX™ passive reference dye, taq polymerase, free nucleotides and MgCl₂ in an optimized buffer solution.
The PCR Cycles are as follows:
Step 1: denaturation: 15 min at 94° C.
Step 2: 10 cycles of 20 seconds at 94° C., 60° secs at 65-57° C. (−0.8° C. par cycle)
Step 3: 35 cycles of 20 seconds at 94° C., 60° secs at 57° C.
Step 4: 15° C.

Alleles fluorescence has been detected on Pherastar Plate Reader and interpretation into FAM/VIC allele has been conduct with KlusterCaller software with predefined cluster assignation parameters.

2. Marker Assisted Introgression of Powdery Mildew Resistance from PM1 Accession in Charentais Melon

A breeding scheme was instituted using QTLs in order to develop a melon line with a high level of resistance to different races of P. xanthii. In an effort to avoid the genetic drift of such a melon line by introducing QTLs from the melon accession PM1 (FIG. 1E), molecular markers were used to introgress resistance QTLs and to break linkage with undesirable agronomic traits (especially pale and soft flesh at maturity, big cavity, high yellowing/orange rind at maturity and low level of sugar (1⁰° Brix)) known to be associated with the QTLs providing Powdery Mildew (PM) resistance.

The Charentais elite line C (FIG. 1B) was crossed with the melon accession PM1, a Cucumis melo sp. Momordica plant known to be resistant but having undesirable agronomic traits. The resultant F1 seeds were germinated, plants grown from the germinated seeds, and the resultant plants were selfed to produce F2 seeds/plants for further selection and breeding. Three hundred (300) F2 plants have been submitted to PM leaf disk assay with a local isolate Px3-5. Among the resistant plants, a set of around 50 markers microsatellites (SSRs) have been used to select F2 plants. These markers have been centered on LG2 and LG5, which are known from the literature, as bearing the Powdery Mildew resistance QTLs in Cucumis melo sp. Momordica species. The F2 plants which harbor the QTLs on LG2 and LG5 suspected to be linked to the PM resistance, at homozygous stage, have been selected. Six F2 plants out the 300 the F2 plants were selected and were backcrossed with the Charentais elite line C to obtain BC1 seeds/plants. At this heterozygous level, no PM pressure has been done. Only a phenotypic evaluation of the BC1 plants has been performed. Fruits of the BC1 plants have been observed internally and externally. Plants having round or not too oblong fruits with a clear bark color and netted as regularly as possible and firm, colorful, sweet and non-vitreous fruits with a storage for 5 days at 12° C. and 2 days at room temperature after opening are retained.

The same approach has been adopted at most of the following selection stage, where the plants have been selected mostly on their agronomic features, and plants showing any necrosis factor have been eliminated.

Moreover, a genotyping with the around 50 microsatellites (SSRs) allowing to calculate the % of the recurrent genome on these BC1 and the size of the introgressed fragments has been performed. One population of BC1 plants having the smaller introgression fragments, a clear bark and a nice dark green drawing, a dark orange flesh, firm and sweet, and a little elongated shape possible has been selected. BC1 plants from such a population were selfed to produce F2BC1 seeds/plants. Two hundred and thirty-six (236) F2BC1 plants have been submitted to PM test with a local isolate Px 3-5. Then the 67 resistant plants have been screened with the SSRs in order to select plants which harbor the shorter QTLs/introgression from PM1 on LG2 and LG5 at homozygous or heterozygous stage. Five F2BC1 plants out of the 236 F2BC1 plants were selected and were selfed in order to produce seeds/plants of F3BC1 or backcrossed with the Charentais elite line C to obtain BC2 seeds/plants. F3BC1 progenies are submitted to PM individual races Px 1/2/3/5/3-5 by a leaf disk assay to validate the wide range spectra of the selected plants. All the tested plants had a good level of resistance to all these races.

One hundred and seventy (170) BC2 plants have been screened with the same SSRs to keep shorter fragments of PM1 on the carrier linkage groups (LG) and higher recurrent genome on the non-carrier linkage groups. Fifteen BC2 plants out of the 170 BC2 plants were selected and were selfed in order to produce seeds/plants of F2BC2. F2BC2 plants were selfed to produce F3BC2 seeds/plants that were backcrossed with the Charentais elite line C to obtain BC3 seeds/plants. BC3 plants were selfed to produce F2BC3 seeds/plants that were selfed to produce F3BC3 seeds/plants. F3BC3 plants were also selfed to obtain F4BC3 seeds/plants. Thirty-three (33) F4BC3 progenies were analyzed with 3 SSRs on each carrier LG (CMAG36, NR39 and CMBR120 for LG2, and CNTAAN128, NR2 and LG5-M2 for LG5) after an artificial PM leaf disk assay with individual races Px 1/2/3/5/3-5. Such an analysis demonstrated that the QTLs of resistance from PM1 are indeed on LG2 and LG5 and have been fixed by the breeding scheme, essentially based on phenotypic selection. Plants of the F4BC3 were selected and crossed with line F (FIG. 1C), to produce BC4 seeds/plants. Such BC4 plants have been selfed 4 times in order to produce F5 plants that can be used as a donor line for QTLs of resistance to PM on LG2 and LG5. At this stage, the linkage with the necrosis factor on LG2 was however still present, responsible for an hyper reaction by necrosis of the plants in contact with water drops.

3. Marker Assisted Introgression of Scab Resistance from SC1 Landrace in Charentais Melon

A breeding scheme was instituted using QTLs present in wild species, in order to develop a melon line with a high level of resistance to C. cucumerinum. In an effort to avoid the genetic drift of such a wild melon line by introducing QTLs from the melon accession SC1 (FIG. 1D), molecular markers were used to introgress resistance QTLs and to break linkage with undesirable agronomic traits (especially pale orange and soft flesh at maturity, big cavity, ribby shape, high yellowing rind at maturity and low level of sugar (10° Brix)) associated with the resistance.

The Charentais elite line A (FIG. 1A) was crossed with the melon accession SC1. The resultant F1 seeds were germinated, plants grown from the germinated seeds, and the resultant plants were backcrossed with the Charentais elite line A to produce BC1 seeds/plants for further selection and breeding. Ninety-six BC1 plants were submitted to Scab test (see example 1.1). Among the resistant plants, a set of 36 molecular markers spread among the genome, and capable of differentiating genomic sequences from both sources, have been used to select BC1 plants with the highest elite genome ratio, i.e. the least genomic sequences introgressed from SC1. Among the dozen of selected BC1 plants, the 3 best ones have been selected based on fruit assessment and have been selfed to produce BC1i1 seeds/plants. Twenty-four BC1i1 plants have been submitted to Scab test, and the obtained data associated with phenotypic evaluation of BC1 plants and fruits allowed to select the best 2-3 best BC1 plants and the BC2 progenies to follow. Accordingly, the best 2-3 BC1 plants were backcrossed with the Charentais elite line A to produce BC2 seeds/plants. Ninety-six BC2 plants were submitted to Scab test. Among the resistant plants, the same set of 36 molecular markers has been used to select BC2 plants with the highest elite genome ratio. The selected BC2 plants were selfed to produce BC2I1 seeds/plants. The process was pursued by the inventors with one additional backcross, with an important selection pressure applied on the agronomical features of the plants to be selected for the further steps, especially fruit form, flesh color and firmness, size of the cavity and bark color at maturity. Moreover, for the 3^{rd} backcross, alternative hybrid lines have been tested as recurrent parent, in order to promote diversification of the scab resistance, while simultaneously bringing earliness, netting and internal quality of the flesh. Two additional selfings were carried out maintaining the selection pressure on the scab resistance and agronomical features. One specific line was retained to be combined with the material bearing the introgressed QTLs from PM1 providing powdery mildew resistance as selected in example 2.

4. Combination of Powdery Mildew Resistance from PM1 Accession and Scab Resistance from SC1 Accession in Charentais Melon

From a cross between one line coming from the PM1 accession breeding scheme (see example 2) and one line from the SC1 breeding scheme (see example 3), F2 plants were generated. Four hundred F2 plants have been submitted to a SCAB test and then analyzed by markers on LG5 to look for potential recombinant events likely to break the linkage with the sequences responsible for the poor quality of fruits, supposed to be found essentially on LG5. Nine F2 plants were kept based on the potential recombinant event and their behavior in the SCAB test. Selection has been pursued on F3 progenies, submitting 200 F3 plants/progeny through a SCAB test, and using markers on LG5 and LG2 on the plants identified as resistant, approximately 50% of the plants. Between 13 to 16 F3 plants per progenies were kept. In each family, the external and internal fruit qualities, the necrotic behavior of each F3 plants and their PM level in greenhouse were assessed. The F4 offsprings of the retained F3 plants have been tested by a race to race PM test and a ZYMV test. The results obtained by the inventors showed that the necrotic reaction of F3 PM resistant plant is closely linked to the ZYMV locus. The less necrotic F3 plants are either susceptible to ZYMV or in disjunction. In the opposite, all the ZYMV resistant plants are really necrotic.

The selection scheme has thus been pursued on two specific F4 progenies, respectively susceptible to ZYMV (progeny 1607/003) and in disjunction (progeny 1607/007). The PM tests revealed a slightly higher level in the second one (see table A) with the same necrotic pattern, namely absence of necrotic pattern. The F5 progeny numbered 570 (issued from the F4 1607/007) has been selected for further test on SCAB resistance. Specifically, 20 plants of the F5 progeny numbered 570 (from the F4 1607/003) have been tested for SCAB resistance, as well as two resistant controls and two susceptible controls.

The results are reported below in table B and demonstrate a very low disease index for these plants in the SCAB test.

Seeds of this F5 progeny, named MTYVVC721, obtained by self-pollination, resistant to PM and SCAB, were deposited at the NCIMB, under accession number NCIMB 43317 on 13 Dec. 2018. These plants (see illustrative plants on FIG. 1F) have phenotypic features essentially identical to those of commercial melon lines (see illustrative plants on FIGS. 1A, 1B and 1C).

TABLE A
results of the PM detached leaf test- Susceptibility score.

| | GC | | Px 1 | | Px 2 | | Px 3 | | Px5 | | Px3-5 | |
|---|---|---|---|---|---|---|---|---|---|---|---|---|
| | Lect1 | Lect2 | Lect1 | Lect2 | Lect1 | Lect2 | Lect1 | Lect2 | Lect1 | Lect2 | Lect1 | Lect2 |
| | (R1/ | (R1/ | (R1 | (R1/ | (R1/ | (R1/ | (R1/ | (R1/ | (R1/ | (R1/ | (R1/ | (R1/ |
| CODE | R2) | R2) | /R2) | R2) | R2) | R2) | R2) | R2) | R2 | R2) | R2) | R2) |
| 1607/003 | 0/0 | 0/0 | 0/0 | 0/0 | 0/0 | 0/0 | 0/0 | 0/0 | 0/0.07 | 0/0.07 | 0/ | 0.2/ |
| (mean of 15 | | | | | | | | | | | 0.27 | 2.2 |
| plants) | | | | | | | | | | | | |
| 1607/007 | 0/0 | 0/0 | 0/0 | 0/0 | 0/0 | 0/0 | 0/0 | 0/0 | 0/0 | 0/0 | 0/0 | 0/0.14 |
| (mean of 15 | | | | | | | | | | | | |
| plants) | | | | | | | | | | | | |
| PM1 | 0/0 | 0/0 | 0/0 | 0/0 | 0/0 | 0/0 | 0/0 | 0/0 | 0/0 | 0/0 | 0/0 | 0/0 |
| Vedrantais | 3/1 | 7/7 | 5/5 | 7/7 | 7/7 | 7/9 | 5/7 | 7/9 | 5/3 | 5/3 | 5/3 | 5/7 |
| Nantais | 0/0 | 0/0 | 5/7 | 7/7 | 7/5 | 7/7 | 5/7 | 7/9 | 5/5 | 7/5 | 5/1 | 7/5 |
| Oblong | | | | | | | | | | | | |
| PMR45 | 0/1 | 0/1 | 0/0 | 0/0 | 5/5 | 7/9 | 5/7 | 7/ND | 5/5 | 5/5 | 1/0 | 5/0 |
| PMR45 | 0/0 | 1/0 | 0/0 | 0/0 | 5/5 | 5/5 | 5/ND | 7/ND | 5/3 | 7/3 | 3/0 | 3/1 |
| Edisto 47 | 0/0 | 0/0 | 0/0 | 0/0 | 0/0 | 0/0 | 0/0 | 0/0 | 5/5 | 5/7 | 3/0 | 5/3 |
| Edisto 47 | 0/0 | 0/0 | 0/0 | 0/0 | 0/0 | 0/0 | 0/0 | 0/0 | 5/5 | 5/5 | 3/0 | 5/0 |
| PMR5 | 0/0 | 0/0 | 0/0 | 0/0 | 0/0 | 0/0 | 1/1 | 0/0 | 0/0 | 0/0 | 1/0 | 5/0 |
| PMR5 | 0/0 | 0/0 | 0/0 | 0/0 | 0/0 | 0/0 | 1/3 | 0/0 | 0/0 | 0/0 | 1/0 | 3/0 |

Susceptibility scores results obtained by the PM detached leaf test (see example 1.2), applied on 15 plants of the two progenies 1607/003 and 16/007, as well as on resistant controls, namely PM1 the introgression partner, PMR5 (two plants, controls for some strains of Podosphaera xanthii) and on susceptible controls, namely Védrantais, Nantais oblong. Plants known to be resistant to at least one of the strains have also been tested (Edisto 47, PMR45). ND=non-determinable.

TABLE B
results of the SCAB test - disease scores and disease index

| | Resistance | Resistance | Resistance | Resistance | Resistance | |
|---|---|---|---|---|---|---|
| | Score 9 | Score 7 | Score 5 | Score 3 | Score 1 | Disease |
| Plants | (nb of plants) | (nb of plants) | (nb of plants) | (nb of plants) | (nb of plants) | Index |
| F5 progeny | 15 | | 5 | | | 0.89 |
| 570 of | | | | | | |
| 1607/007 | | | | | | |
| Susceptible | | | | 1 | 1 | 0.17 |
| control | | | | | | |
| Resistant | 2 | | | | | 1.00 |
| control | | | | | | |

The disease index is calculated as follows:

DI=[(0×Nb of plants have a resistance score 1)+(3×Nb of plants having resistance score 3)+(5×Nb of plants having resistance score 5)+(7×Nb of plants having resistance score 7)+(9×Nb of plants having resistance score 9)]/(9×total number of plants).

These results confirm that the seeds deposited at the NCIMB are resistant to PM and resistant to SCAB. Resistance to aphids provided by the Vat gene has also been confirmed. These plants also have a commercially acceptable fruit quality and aspect (see FIG. 1F), and do not have any necrotic phenotype linked to the Zym gene.

### 5. Development of Specific Markers

Using a genome scan approach, more than 4500 SNP markers evenly distributed along the genome and covering well the 12 melon chromosomes (corresponding to the 12 linkage groups) have been genotyped on a panel of lines. This panel included ZYMV resistant lines, PM resistant lines, SCAB resistant lines and susceptible lines, together with the original SCAB and PM resistant donors, i.e. PM1 and SC1, the susceptible lines at the origin of the breeding process, i.e. elite lines A and C, and the final breeding line pyramiding the SCAB and PM QTL without impacting the fruit quality (MTYVVC721 and progeny thereof). Data analysis allow the identification of specific marker haplotypes for the SCAB resistance QTL on the LG2 and on the LG5 and for the PM resistance QTL on the LG2 and on the LG5.

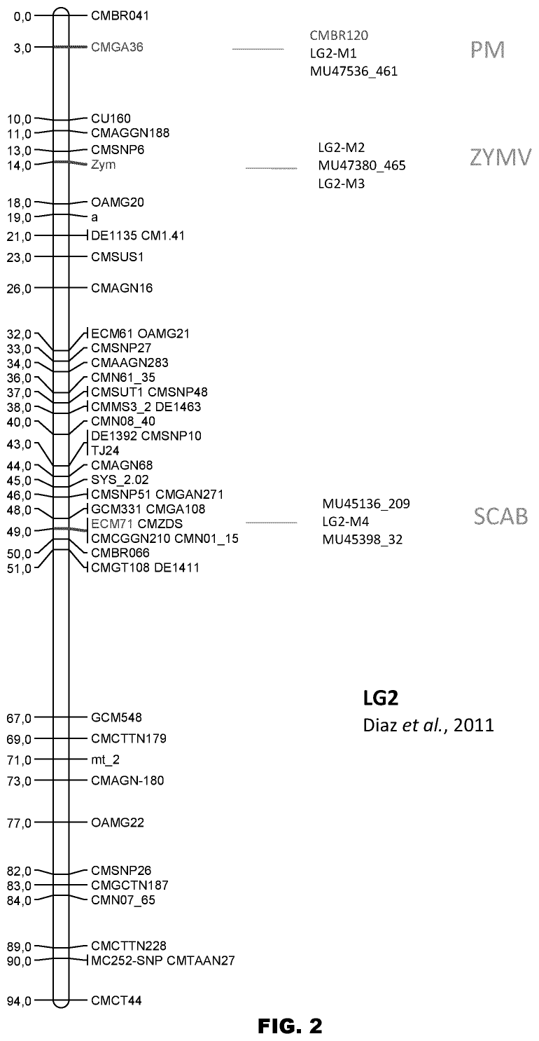

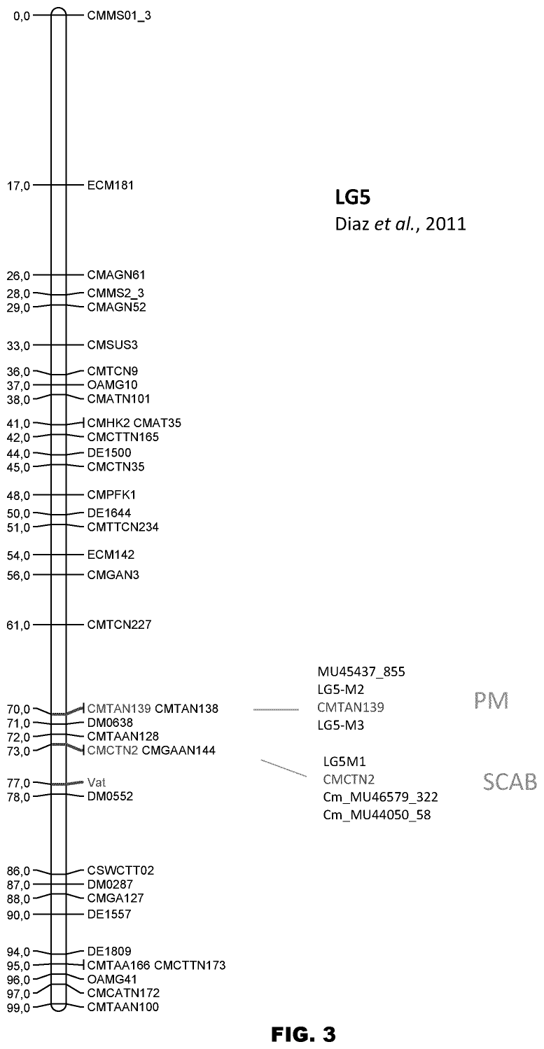

The flanking markers, as well as further markers within the regions delimited by these flanking markers are reported in Table C, for the SCAB resistance QTL on LG2 and LG5 and for the PM resistance QTL on LG2 and LG5. Moreover, the positions of the markers on the genome assembly of Melon (DHL92) version 3.6.1 (Garcia-Mas et al, 2012) and available at the following address http://cucurbitgenomics.org/organism/18 are also reported in table C. The position of said markers with respect to some of the markers disclosed in Diaz et al, 2011, are also illustrated in FIG. 2 (LG2) and FIG. 3 (LG5).

Moreover, by following the ZYMV gene, known to be in the vicinity of the PM resistance QTL on LG2, the inventors have confirmed that this gene is no longer present in the plants having a genome corresponding to the deposited seeds. They have thus confirmed at the molecular level the recombination events observed at the phenotypic level, i.e. loss of ZYMV gene by recombination gives rise to plants having no necrosis symptoms. The markers used for detecting the presence or the absence of the ZYMV gene, as well as their physical positions on the same version of the melon genome are also given in Table C. SSR and SNP genotyping have been carried out as detailed in example 1.

Table D details, for the SNP markers of table C, the polymorphism as such, the allele linked to presence of the resistance QTL, the sequence surrounding the informative polymorphism (i.e. the polymorphic nucleotide and the 3′ and 5′ flanking sequences), and the corresponding SEQ ID No in the accompanying sequence listing.
Table E reports, for the SSR markers of table C, the forward and reverse primers to be used for amplifying the microsatellite, as well as the length of the amplified fragment representative of the presence of the resistance QTL (PM or SCAB resistance QTL).

Table F reports, for the makers of tables D and E, the alleles which have been found in the panel of lines which have been genotyped.

Table G describes the primers which have been used by the inventors for detecting the alleles of the SNPs as disclosed in the invention.

TABLE C
List of markers.
The columns indicate the name of the markers,
the type of marker (SSR or SNP), the type of
resistance which is marked, the Linkage Group
and the position on the melon genome version
3.6.1 on this LG for each marker (corresponding
to the position of the polymorphic nucleotide
for SNP; for the microsatellite, the position
given is with respect to the sequence of the
primers used for the amplification) and whether
the marker is a flanking marker of the resistance QTL.

| Name of | Type of | Type of | Linkage | Position | Flanking |
|---|---|---|---|---|---|
| the marker | SNPs | resistance | Group | (bp) | marker |
| CMBR120 | SSR | PM | LG2 | Around | yes |
| | | | | 787 830 | |
| LG2-M1 | SNP | PM | LG2 | 788 941 | |
| MU47536_461 | SNP | PM | LG2 | 843 667 | yes |
| LG2-M2 | SNP | ZYMV | LG2 | 933 153 | yes |
| MU47380_465 | SNP | ZYMV | LG2 | 936 660 | |
| LG2-M3 | SNP | ZYMV | LG2 | 968 880 | yes |
| MU45136_209 | SNP | SCAB | LG2 | 6 454 350 | yes |
| LG2-M4 | SNP | SCAB | LG2 | 7 036 176 | |
| MU45398_32 | SNP | SCAB | LG2 | 7 476 525 | yes |
| LG5-M1 | SNP | SCAB | LG5 | 25 228 049 | yes |
| CMCTN2 | SSR | SCAB | LG5 | Around | |
| | | | | 25 298 010 | |
| MU46579_322 | SNP | SCAB | LG5 | 25 388 402 | |
| MU44050_58 | SNP | SCAB | LG5 | 25 424 775 | yes |
| MU45437_855 | SNP | PM | LG5 | 25 257 091 | yes |
| LG5-M2 | SSR | PM | LG5 | Around | |
| | | | | 26 038 930 | |
| CMTAN139 | SSR | PM | LG5 | | |
| LG5-M3 | SNP | PM | LG5 | 26239817 | yes |

TABLE D
SNPs markers.
This table reports the different SNP markers identified, for the different
resistance, the sequence of the markers, including the polymorphic nucleotide. The
column entitled ″Polym R/S″ indicates the two alleles of the polymorphic nucleotide,
the allele linked to the resistance (″resistant allele″) is mentioned first, the
second one being the allele representative of susceptibility.

| | | | Polym | SEQ |
|---|---|---|---|---|
| Marker | QTL | Sequence | R/S | ID |
| LG2-M1 | PM | TTAATGGAATGACACAGATCAATGTTCTCATCTGTTGCCCTC | T/C | SEQ |
| | | ATGGCTTCTGATACTCGTTCTGATAT[T/C]CCGGGGCCCAAA | | ID NO: |
| | | TGAGGATTGTATGTTCTAACCCATTCTTCGTGATTGATTTCG | | 26 |
| | | AATTC | | |
| MU47536_461 | PM | GGTAACATGTGGGAGATTGATGTACAAGATTTTGATAATGTG | T/A | SEQ |
| | | ATTGATAC[A/T]AACATCAAAGGCTCAGCTAATATTCTTCGTC | | ID NO: |
| | | ACTTCATCCCTCTTATGAT | | 30 |
| LG2-M2 | ZYMV | GAATTCGAGGGAGTTGTGAGCTCATGAGACGAAGCGTTTGA | A/C | SEQ |
| | | TTCATGTGAC[A/C]CCGGGTTTCCAAGAGTAACAATTTCTCG | | ID NO: |
| | | AGCTTTAGATGAWWARAWTCTCACAATGAAAWAGAAGATTT | | 50 |
| | | GGTAAAATATATCCAAGAATTGGAAYTKGGATTGTCGATTGT | | |
| | | CCATTGGAATGTTTTTGGDRGGARRGGGKGKTGRATTAMW | | |
| MU47380_465 | ZYMV | TTATCCTCCATATTTAATGAATTAGTCGATCGAGTGAGCATG | A/G | SEQ |
| | | GATGGGAA[A/G]TTTCTACAGGAATCACTATCCAGAACTAAA | | ID NO: |
| | | AAAGGAGATGCTTTTACGTC | | 54 |
| LG2-M3 | ZYMV | TYYGACAARTTTTGTTCTCTACATTAGATTTCCCTTCATGATG | G/A | SEQ |
| | | TTGTAGT[A/G]ACATGGAGGGTTGTTTCCTTTAAAAATGTACA | | ID NO: |
| | | GGTTGGTAACATTAAGGC | | 58 |
| MU45136_209 | SCAB | TGCTTGCTGCCGGCCTCCCTGGCGGCCGCGTGAATTTTATT | C/T | SEQ |
| | | TAACGGCCCAATCGGCCCACACATTGCATCAAAAAAAAATAT | | ID |
| | | TTCCCAAGTGTTTCTTAGCATATATTTGTTGTGTTCAAAGAAA | | NO: 1 |
| | | CCAAAATATCAAAAGAGGTGCGCTATTATTTTTTTATCATCG | | |
| | | GTTCTTGTCTCAAGAAGGAAAAAAAAATGTCACTCTTTCT[T/ | | |
| | | C]AAGTCTTGCAGCTCCAAGAAATTTGTTGGTGATCATTCATTA | | |
| LG2-M4 | SCAB | CATGGAGTGCCGCATACGGTTGATACTTTGGTTTTCACACC | A/G | SEQ |
| | | ATTTGTAAGTTTGAACTTT[A/G]CAAACACTAAAGAACTCGTT | | ID NO: |
| | | ATCATACGTGCTTGCATTACCTTTCTTGTGATAAATGTGT | | 5 |
| MU45398_32 | SCAB | AGAATAACAAAACAGGGTTGTTCCGCTTTAC[T/C]TCAAATA | C/T | SEQ |
| | | GCAACAAAGAAGAAGAAGAAGAAGACGATTAGAAGAGAAAG | | ID NO: |
| | | ATGGAAGCGAAGGTCAGCAAATTCTTAGGCTCCGTTTCCAA | | 9 |
| | | TTTCTTCTCCGGCGGCGATCACATACCATGGTGCGATCGCG | | |
| | | ACGTCGTTGCCGGTTGCGAAAGAGAGGCTGCTGAGGCCGA | | |
| | | AAAGAGCTCATCTGGTGAGCTTTTGAAGGAAAGCATTATGC | | |
| | | GTTTATCT | | |
| LG5-M1 | SCAB | TCCATCCATCTTTTCCAGTTTTGTGGAGTTCATTA[T/C]GATA | C/T | SEQ |
| | | CAATTTCGCCGATCTACTTTTCTTTACAT | | ID NO: |
| | | | | 13 |
| MU46579_322 | SCAB | GAAGACTCCGGTAGCTAAAGAAGGCGGTTCCGATCCTCACT | C/T | SEQ |
| | | GGAACTATC[T/C]CATGAAGTTCACAGTCGATGAGGCTGCCT | | ID NO: |
| | | TGCAGAACAATCGACTCAATC | | 16 |
| MU44050_58 | SCAB | GAACCCAAAAAGCTTGGGGGTTTCGTAGCTTCGTAGCGTTG | C/T | SEQ |
| | | CTTTCATGGCGAGCTT[T/C]GTTTCTTTGAGCTTCTCCGTTCC | | ID NO: |
| | | ACAGCTCACTCTCAAGGAAATCCCACCATGGAAATTGGCTA | | 20 |
| | | AAGCTACGGTAGTTTCAAGGCGAGCTGGAACAGCTTTGAAG | | |
| | | CTTGTGCTACCCTGTTATTCAAAGAAATCCATATCTATGAATT | | |
| | | GGGAAAACTTTCGATCTGTACGGTTAACTTCAAGACCTTTTT | | |
| | | GGG | | |
| MU45437_855 | PM | CTGAATCATACCAATTGTAATTAAAGTTTCTGTGTATTAAATC | A/G | SEQ |
| | | TGAACTC[A/G]AGGCTTTTCCTCTATATGTGCCTTCGAAACG | | ID NO: |
| | | TGCTCTGTAACTTGATTTT | | 34 |
| LG5-M3 | PM | ATTTCAAGAGAGTTCTTTCTAACGGGCATTGAGATTCCMGAA | T/C | SEQ |
| | | TGGTTCAG[T/C]TATAAGTCTACTACAAATTCTGTGACTGCTA | | ID NO: |
| | | GCATTCGTCACTATCCAGA | | 42 |

TABLE E
SSR Markers
This table reports the different SSR markers identified, for the
different resistance, the sequence of two primers which can be used for
amplifying the microsatellite region, as well as the length of the
amplified fragment, representative of the presence of the resistance QTL.

| Marker | Trait | Forward primer | Reverse primer | Length |
|---|---|---|---|---|
| CMBR120 | PM | CTGGCCCCCTCCTAAACTAA | CAAAAAGCATCAAAATGGTTG | 165 bp |
| | | (SEQ ID NO: 24) | (SEQ ID NO: 25) | |
| CMCTN2 | SCAB | CTGAAAGCAGTTTGTGTCGA | AAAGAAGGAAGAGGCTGAGA | 195 bp |
| | | (SEQ ID NO: 14) | (SEQ ID NO: 15) | |
| LG5-M2 | PM | CACTTTCTAAATAGTTTGGAAA | GAGAATGTCTCTTTATCTAC | 178 bp |
| | | AGAG | (SEQ ID v39) | |
| | | (SEQ ID NO: 38) | | |
| CMTAN139 | PM | CGTAGAAGACACACATAATG | GAACTAGAACCACAAATCAC | 134 bp |
| | | (SEQ ID NO: 40) | (SEQ ID NO: 41) | |

TABLE F
genotyping of the parent lines and deposited plants, with respect to the markers
identified

| Patent marker | | | | | | |
|---|---|---|---|---|---|---|
| code | trait | PM1 | Line A | SC1 | MTYVVC721 | Vedrantais |
| CMBR120 | PM | 165/165 | 172/172 | 163/163 | 165/165 | 163/163 |
| LG2-M1 | PM | T/T | T/T | T/T | T/T | T/T |
| MU47536_461 | PM | T/T | T/T | T/T | T/T | T/T |
| LG2-M2 | ZYMV | A/A | C/C | C/C | C/C | C/C |
| MU47380_465 | ZYMV | A/A | G/G | G/G | G/G | G/G |
| LG2-M3 | ZYMV | G/G | A/A | A/A | A/A | A/A |
| MU45136_209 | SCAB | T/T | C/C | T/T | T/C | T/T |
| LG2-M4 | SCAB | G/G | A/A | G/G | A/A | G/G |
| MU45398_32 | SCAB | T/T | C/C | T/T | C/C | T/T |
| LG5-M1 | SCAB | T/T | T/T | C/C | C/C | T/T |
| CMCTN2 | SCAB | 182/182 | 191/191 | 195/195 | 195/195 | 191/191 |
| MU46579_322 | SCAB | T/T | T/T | C/C | C/C | T/T |
| MU44050_58 | SCAB | T/T | C/C | C/C | C/C | C/C |
| MU45437_855 | PM | A/A | G/G | A/A | A/A | G/G |
| LG5-M2 | PM | 178/178 | 172/172 | 176/176 | 178/178 | 172/172 |
| CMTAN139 | PM | 134/134 | 138/138 | NA | 134/134 | 138/138 |
| LG5-M3 | PM | T/T | C/C | C/C | T/T | T/T |

TABLE G
Primers used by the inventors for detecting the alleles of the SNP,
using the KASpar technology and corresponding SEQ ID number.

| | | Forward primer/ | Forward primer/ | |
|---|---|---|---|---|
| marker | trait | Susceptible allele | Resistant allele | Reverse primer |
| Cm_MU45136_209 | Scab | ACAAATTTCTTGGAG | CAAATTTCTTGGAGC | TATCATCGGTTCTT |
| SEQ ID NO: 1 | disease | CTGCAAGACTTA | TGCAAGACTTG | GTCTCAAGAAGGAAA |
| | | SEQ ID NO: 2 | SEQ ID NO: 3 | SEQ ID NO: 4 |
| LG2-M4 | Scab | TTCACACCATTTGTAA | GTTTTCACACCATTTG | GCACGTATGATAAC |
| SEQ ID NO: 5 | disease | GTTTGAACTTTG | TAAGTTTGAACTTTA | GAGTTCTTTAGTGTT |
| | | SEQ ID NO: 6 | SEQ ID NO: 7 | SEQ ID NO: 8 |
| Cm_MU45398_32 | Scab | CAAAACAGGGTTGTT | AAAACAGGGTTGTTC | CGTCTTCTTCTTCT |
| SEQ ID NO: 9 | disease | CCGCTTTACT | CGCTTTACC | TCTTCTTTGTTGCTA |
| | | SEQ ID NO: 10 | SEQ ID NO: 11 | SEQ ID NO: 12 |
| Cm_MU46579_322 | Scab | TCCGATCCTCACTGG | CCGATCCTCACTGGA | CAGCCTCATCGACT |
| SEQ ID NO: 16 | disease | AACTATCT | ACTATCC | GTGAACTTCAT |
| | | SEQ ID NO: 17 | SEQ ID NO: 18 | SEQ ID NO: 19 |
| Cm_MU44050_58 | Scab | GCGTTGCTTTCATGG | CGTTGCTTTCATGGC | CTGTGGAACGGAG |
| SEQ ID NO: 20 | disease | CGAGCTTT | GAGCTTC | AAGCTCAAAGAA |
| | | SEQ ID NO: 21 | SEQ ID NO: 22 | SEQ ID NO: 23 |
| LG2-M1 | PM | CCTCATTTGGGCCCC | AATCCTCATTTGGGC | TCATGGCTTCTGAT |
| SEQ ID NO: 26 | | GGG | CCCGGA | ACTCGTTCTGATAT |
| | | SEQ ID NO: 27 | SEQ ID NO: 28 | SEQ ID NO: 29 |
| Cm_MU47536_461 | PM | ATGTACAAGATTTTGA | ATGTACAAGATTTTGA | CGAAGAATATTAGC |
| SEQ ID NO: 30 | | TAATGTGATTGATACA | TAATGTGATTGATACT | TGAGCCTTTGATGTT |
| | | SEQ ID NO: 31 | SEQ ID NO: 32 | SEQ ID NO: 33 |
| Cm_MU45437_855 | PM | AAAGTTTCTGTGTATT | AATTAAAGTTTCTGTG | CAGAGCACGTTTC |
| SEQ ID NO: 34 | | AAATCTGAACTCG | TATTAAATCTGAACTCA | GAAGGCACATAT |
| | | SEQ ID NO: 35 | SEQ ID NO: 36 | SEQ ID NO: 37 |
| LG5-M3 | PM | CAGTCACAGAATTTG | CAGTCACAGAATTTG | AGAGTTCTTTCTAA |
| SEQ ID NO: 42 | | TAGTAGACTTATAG | TAGTAGACTTATAA | CGGGCATTGAGATT |
| | | SEQ ID NO: 43 | SEQ ID NO: 44 | SEQ ID NO: 45 |
| LG2-M2 | ZYMV | ACGAAGCGTTTGATT | GACGAAGCGTTTGAT | CTAAAGCTCGAGAA |
| SEQ ID NO: 50 | | CATGTGACC | TCATGTGACA | ATTGTTACTCTTGGAA |
| | | SEQ ID NO: 51 | SEQ ID NO: 52 | SEQ ID NO: 53 |
| MU47380_465 | ZYMV | CTGGATAGTGATTCC | GTTCTGGATAGTGAT | GAATTAGTCGATCG |
| SEQ ID NO: 54 | | TGTAGAAAC | TCCTGTAGAAAT | AGTGAGCATGGAT |
| | | SEQ ID NO: 55 | SEQ ID NO: 56 | SEQ ID NO: 57 |
| LG2-M3 | ZYMV | ATTAGATTTCCCTTCA | AGATTTCCCTTCATG | TGTACATTTTTAAA |
| SEQ ID NO: 58 | | TGATGTTGTAGTA | ATGTTGTAGTG | GGAAACAACCCTCCAT |
| | | SEQ ID NO: 59 | SEQ ID NO: 60 | SEQ ID NO: 61 |

6. Genetic Modification of C. melo Seeds by Ethyl Methane Sulfonate (EMS)

Seeds of C. melo plants are to be treated with EMS by submergence of approximately 2000 seeds into an aerated solution of either 0.5% (w/v) or 0.7% EMS for 24 hours at room temperature.

Approximately 1500 treated seeds per EMS dose are germinated and the resulting plants are grown, preferably in a greenhouse, for example, from March to September, to produce seeds.

Following maturation, M2 seeds are harvested and bulked in one pool per variety per treatment. The resulting pools of M2 seeds are used as starting material to identify the individual M2 seeds and the plants resistant to Scab, aphids and Powdery Mildew (PM).

### REFERENCES

Burger et al., 2010, Horticultural Reviews, 36, 165-198
Diaz et al., 2011, “A consensus linkage map for molecular markers and Quantitative Trait Loci associated with economically important traits in melon (Cucumis melo L.)”. BMC Plant Biology 2011 11:111.
Dogimont et al., Cucurbitaceae 2008, Proceedings of the IXth EUCARPIA meeting on genetics and breeding of Cucurbitaceae;
Dogimont et al., 2014, The Plant Journal, 80, 993-1004
Fazza et al., 2013, Crop Breeding and Applied Biotechnology, 13:349-355
Fukino et al., 2008, Theor. Appl. Genet., 118(1):165-75
Garcia-Mas et al, 2012, PNAS 109(29):11872-11877.
Needleman and Wunsch, 1970 J. Mol. Biol. 48:443-453)
Perchepied et al., 2005, The American Phytopathological Society, Vol. 95(5):556-565
Pitrat and Lecoq, 1982, Agronomie, 2:503-508
Pitrat and Lecoq, 1984, Euphytica, 33(1):57-61,
US20140059712

1. A Cucumis melo (C. melo) plant that is resistant to Scab disease, aphids and powdery mildew, wherein said plant:
comprises:
(i) at least one QTL conferring resistance to Scab disease, wherein said at least one QTL is present on linkage group 2 (LG2) and/or linkage group 5 (LG5),
(ii) at least one QTL conferring resistance to PM, wherein at least one QTL is present on LG2 and/or LG5, and is different from said one or more QTL in (i), and
(iii) the Vat gene analog associated to aphids resistance on LG5, and
has a commercially acceptable fruit quality.

2. The C. melo plant according to claim 1, wherein said plant does not have any necrotic phenotype linked to the Zym gene.

3. The C. melo plant according to claim 1, wherein:
(i) said QTL conferring resistance to Scab disease that is present on LG2 is located within a chromosomal region that is delimited by marker Cm_MU45136_209 and marker Cm_MU45398_32, and
(ii) said QTL conferring resistance to Scab disease that is present on LG5 is located within a chromosomal region that is delimited by marker LG5-M1 and marker Cm_MU44050_58,
(iii) said QTL conferring resistance to PM that is present on LG2 is located within a chromosomal region that is delimited by marker CMBR120 and marker Cm_MU47536_461, and
(iv) said QTL conferring resistance to PM that is present on LG5 is located within a chromosomal region that is delimited by marker Cm_MU45437_855 and marker LG5-M3.

4. The C. melo plant according to claim 1, wherein:
said QTL conferring resistance to Scab disease LG2 is identified by detection of markers Cm_MU45136_209, LG2-M4, and/or Cm_MU45398_32,
said QTL on LG5 conferring resistance to Scab disease is identified by detection of markers LG5-M1, CMCTN2, Cm_MU46579_322, and/or Cm_MU44050_58,
said QTL on LG2 conferring resistance to PM is identified by detection of markers CMBR120, LG2-M1, and/or Cm_MU47536_461,
said QTL on LG5 conferring resistance to PM is identified by detection of markers Cm_MU45437_855, LG5-M2, CMTAN139, and/or LG5-M3, and
the Vat gene analog associated to aphids resistance on LG5.

5. The C. melo plant according to claim 1 wherein said one or more QTLs associated to resistance to Scab disease, aphids and powdery mildew are chosen from those present in the genome of a plant of the line MTYVVC721 (NCIMB accession number 43317).

6. The C. melo plant according to claim 1 wherein said plant is a progeny of a plant of line MTYVVC721 (NCIMB accession number 43317).

7. The C. melo plant according to claim 1 wherein said Scab disease is caused by Cladosporium cucumerinum.

8. The C. melo plant according to claim 1 wherein said powdery mildew is caused by Podosphaera xanthii.

9. A cell of the C. melo plant according to claim 1.

10. A plant part obtained from the C. melo plant as defined in claim 1.

11. The plant part according to claim 10, wherein said plant part is a seed, a fruit, a reproductive material, roots, flowers, a rootstock or a scion.

12. A seed of a C. melo plant, giving rise when grown up to a plant according to claim 1.

13. A hybrid plant of C. melo, obtained by crossing a C. melo plant with the resistant plant according to claim 1.

14. A method for detecting and/or selecting a C. melo plant that is resistant to Scab disease, aphids and powdery mildew, wherein said method comprises the steps of:
(i) detecting the presence of at least one QTL conferring resistance to Scab disease, wherein said at least one QTL is present on linkage group (LG) 2 and/or linkage group 5 (LG5),
(ii) at least one QTL conferring resistance to PM, wherein said at least one QTL is present on LG2 and/or LG5, and is different from said at least one QTL in (i), and
(iii) detecting the presence of the Vat gene analog associated to aphids resistance on LG5, and
(iv) optionally detecting the absence of the necrotic phenotype linked to the Zym gene.

15. (canceled)

16. A method for increasing the number of harvestable Cucumis melo (C. melo) fruit in an environment infested by Scab disease, aphids and Powdery Mildew (PM) comprising growing in said environment C. melo plants resistant to Scab disease, aphids and PM as defined in claim 1, thus increasing the number of harvestable C. melo fruit.

17. A method for protecting a field from infestation and/or spread of Scab disease, aphids and Powdery Mildew (PM), comprising growing in said environment C. melo plants resistant to Scab disease, aphids and PM as defined in claim 1, thus protecting the field from infestation and spread of Scab disease, aphids and Powdery Mildew.

18. (canceled)

19. (canceled)

20. Molecular markers for detecting Cucumis melo (C. melo) plant that is resistant to Scab disease, aphids and Powdery Mildew (PM), wherein said one or more markers is/are located in at least one of the following chromosomal region:
in the chromosomal region delimited on LG2 by marker Cm_MU45136_209 and marker Cm_MU45398_32,
in the chromosomal region delimited on LG5 by marker LG5-M1 and marker Cm_MU44050_58,
in the chromosomal region delimited on LG2 by marker CMBR120 and marker Cm_MU47536_461,
in the chromosomal region delimited on LG5 by marker Cm_MU45437_855 and marker LG5-M3, or
in the Vat gene analog.

21. A container comprising the melon plant as defined in claim 1, the plant part, the seed, or the hybrid plant.

22. A method for the production of C. melo plantlets or plants resistant to Scab disease, aphids and Powdery Mildew (PM), which method comprises:
i. culturing in vitro the isolated cell or tissue of the C. melo plant according to claim 1 to produce C. melo micro-plantlets resistant to Scab disease, aphids and PM, and
ii. optionally further subjecting the C. melo micro-plantlets to an in vivo culture phase to develop into C. melo plant resistant to Scab disease, aphids and PM.

23. A container comprising the seed as defined in claim 12.
